(19) United States
(12) Patent Application Publication
(10) Pub. No.: US 2011/0009351 A1
(43) Pub. Date: Jan. 13, 2011

(54) SCREENING ASSAY TO IDENTIFY CORRECTORS OF PROTEIN TRAFFICKING DEFECTS

(75) Inventors: David Y. Thomas, Montreal West (CA); John Hanrahan, Montreal West (CA); Graeme Carlile, Montreal (CA); Renaud Robert, Montreal (CA)

Correspondence Address: FOLEY AND LARDNER LLP;SUITE 500, 3000 K STREET NW, WASHINGTON, DC, 20007, US

(73) Assignee: Traffick Therepeutics Inc.

(21) Appl. No.: 12/599,246

(22) PCT Filed: May 9, 2008
(86) PCT No.: PCT/CA08/00896

§ 371 Date: Sep. 23, 2010

Related U.S. Application Data
(60) Provisional application No. 60/916,981, filed on May 9, 2007.

(51) Int. Cl. A61K 31/7034; A61K 31/282; A61K 31/015; A61K 31/192; A61K 31/216; A61K 31/55; A61K 31/519; A61K 31/4706; A61K 31/423; A61K 31/165; A61K 31/343; A61K 31/136; A61K 31/352; A61K 31/337; A61K 31/568; A61K 31/7072; A61K 31/285; A61K 31/4184; A61K 31/7032; A61K 31/5375; A61K 31/52; A61K 31/551; A61K 31/445; A61K 31/24; A61K 31/235; A61P 11/00

(52) U.S. Cl. 514/33; 435/7.21; 514/492; 514/763; 514/570; 514/534; 514/217; 514/262.1; 514/313; 514/375; 514/628; 514/469; 514/649; 514/456; 514/449; 514/178; 514/468; 514/50; 514/504; 514/394; 514/25; 514/239.2; 514/263.4; 514/220; 514/315; 514/532

(57) ABSTRACT

The present invention relates to a novel assay or screen for identifying compounds with potential therapeutic value for the treatment of protein trafficking diseases such as Cystic Fibrosis (CF) and nephrogenic diabetes insipidus (NDI). The usual approach involves expressing the mutant form of the gene in cells and assaying function in a multiwell format when cells are exposed to libraries of compounds. Although such functional assays are useful, they do not directly test the ability of a compound to correct defective trafficking of the protein. To address this a novel corrector screening assay for CF has been developed in which the appearance of the mutant protein at the cell surface is measured as the assay output. This assay was used to screen more than 3100 compounds. This novel screening approach to protein trafficking diseases is robust and general, and may enable the selection of molecules that can be translated rapidly to a clinical setting.

The present invention claims priority from U.S. Patent Application Ser. No. 60/916, 981 filed on May 9, 2007, which is hereby incorporated by reference in its entirety.

### FIELD OF THE INVENTION

The invention relates generally to field of biological assays or screens. More specifically, it concerns an assay for identifying compounds based on their ability to enable delivery of a mutant protein to the cell surface in order to correct protein trafficking defects. The invention further comprises molecules that have been identified as effective for this purpose.

### BACKGROUND OF THE INVENTION

The folding and subsequent trafficking of proteins to their correct cellular location is determined by a complex network of chaperones and other components of the secretory pathway. Defective protein folding or trafficking underlies many human pathologies, including cystic fibrosis (CF), nephrogenic diabetes insipidus and congenital long QT syndrome.^{1,2}

Small molecules that can act directly as chemical chaperones for folding proteins or indirectly to enhance the activity of endogenous chaperones would be useful tools for dissecting protein folding and trafficking mechanisms and for the development of therapeutics. The mutations that underlie these diseases are known, and many give rise to proteins that would be functional if they were not recognized by the cellular protein quality control machinery and proteolytically degraded. These facts validate the strategy of developing small molecule correctors.²

Previous studies have searched for agents that inhibit proteasomal degradation, increase the level of protein expression, or enhance its activity (compounds referred to as potentiators).³ While they have generated small molecules that are useful as tools and are potential therapeutics, such approaches may miss potentially valuable classes of molecules.

Cystic fibrosis (CF) is a prototypic disease of protein trafficking. It is an autosomal recessive lethal disorder which occurs with a frequency of one in 2200 live births in North America and Europe, and mainly affects epithelial cells that line the airways, intestine and exocrine tissues.⁴ In CF patients, the airway epithelial surface becomes dehydrated, disrupting the normal mucociliary clearance of inhaled pathogens. This causes recurring infections that produce chronic inflammation leading to fibrosis and a gradual deterioration in lung function that shorten the mean life span of CF patients to about 35 years.⁴

CF is caused by mutations in the cystic fibrosis transmembrane conductance regulator (CFTR) gene,³ which encodes a cAMP stimulated chloride ion channel in the plasma membrane of epithelial cells.^{2,5} Of 1526 documented mutations of the CFTR gene, the most common is a phenylalanine deletion (ΔF508) in the first nucleotide binding domain (NBD). Approximately 90% of CF patients have at least one copy of this variant.⁶

ΔF508-CFTR is retained in the endoplasmic reticulum (ER) and then degraded, however, it can be rescued by incubation at lower temperatures (≦30° C.) or with chemical chaperones such as phenylbutyrate or glycerol.⁷ The rescued protein has a shortened half-life and is less responsive to stimulation by cAMP agonists.⁸ It is believed that recovery of a small fraction of ΔF508-CFTR (6-10%) is sufficient to correct anion transport and provide therapeutic benefit. Hence therapies that even partially correct the effects of this mutation should benefit most CF patients.⁹

It has been reasoned that an assay to directly identify classes of small molecules that promote trafficking of aberrant proteins to their correct cellular destinations would be complementary to screens that are based on functional assays. Such an assay may also be useful for the identification of molecules or drugs suitable for the treatment or alleviation of other respiratory conditions or illnesses, such as Chronic Obstructive Pulmonary Disease (COPD).

There is thus a need for such an assay. The present invention seeks to meet this and related needs.

### SUMMARY OF THE INVENTION

The present invention relates to an assay for identifying compounds based on their ability to enable delivery of a mutant protein to the cell surface.

More specifically, a cell-based assay for monitoring the effect of chemical agents on the trafficking of mutated CFTR to the plasma membrane has been developed. The fourth extracellular loop of CFTR molecule tolerates insertions without significant loss of function. Hence, three HA-tags were inserted into this loop to allow the detection of CFTR on the cell surface by immunofluorescence staining. Tagged CFTR was stably expressed in Baby Hamster Kidney (BHK) cells optimized to the largest difference between negative and positive controls according to preliminary studies. Although functional assays of rescued protein at the cell surface are expected to detect a subset of active compounds, their effectiveness may be limited by the functional properties of the host cell and in the case of CFTR, extensive validation is required to rule out effects on other transport pathways that might affect membrane potential or halide permeability. Focusing on the trafficking defect using a tagged mutant provides a direct and complementary approach for identifying new sets of potentially useful molecules.

Previous high-throughput screens for protein trafficking diseases such as cystic fibrosis have employed functional assays. A rapid and simple microtiter-based screening assay that directly monitors protein trafficking has herein been developed, validated and used. The results provide support for the identification of small molecules that correct the biosynthetic arrest of ΔF508-CFTR using a cell-based trafficking assay that could be extended to other protein trafficking diseases. Screening of the Microsource Discovery (or MSD) small molecule library has demonstrated the potential of this approach, since it identified compounds such as chlorzoxazone that had previously been reported to activate CFTR. The fact that chlorzoxazone was detected in a trafficking correction assay gives the first clue that functional and conformational correction may share a common mechanism. Since the assay identified novel corrector compounds that had not previously been reported, it may be used to identify small molecules that are potentially useful therapeutically for CF and other diseases, including without limitation COPD (chronic or acute bronchitis, emphysema, pneumoconiosis, pulmonary neoplasms, etc.) and nephrogenic diabetis insipidus (NDI).

Other objects, advantages and features of the present invention will become more apparent upon reading of the following non restrictive description of preferred embodiments thereof, given by way of example only with reference to the accompanying drawings.

### BRIEF DESCRIPTION OF THE FIGURES

FIG. 1: Schematic of CFTR protein trafficking. (A) The triple Hemagglutinin tag (HA) and linkers used as an insert into the fourth excellular loop of CFTR after amino acid position 901. (B) A scheme for protein trafficking that demonstrates the 3HA-tag is only accessible after it reaches the cell surface.

FIG. 2: Analysis of the effect of the presence of 3HA tag. (A) Effect of inserting a 3HA-tag on the expression of £F508-CFTR (ΔF508) and wt CFTR (wt) in BHK cells. Cells were cultured for 48 hours at either 37° C. or 27° C., lysed and immunoblotted for CFTR and HA. (B) Effect of the presence of 3HA on the functionality of CFTR for both ΔF508-CFTR (ΔF508) and wt CFTR (wt) in BHK cells cultured at 37° C., as monitored by iodide efflux. Cells were stimulated at time zero with 10 μM forskolin (Fsk) and 50 μM genistein (Gst) (SEM; n=3). (C) Effect of the presence of 3HA on the functionality of CFTR for ΔF508-CFTR (AF) in BHK cells monitored by iodide efflux (as per FIG. 1B) cultured at 27° C. and in the presence of 10% glycerol for 48 hours prior to measurement. Cells were stimulated at time zero with 10 μM forskolin (Fsk) and 50 μM genistein (Gst) (SEM; n=3).

FIG. 3: Demonstration of CFTR trafficking in BHK cells. (A) Changes in the surface expression of both ΔF508-CFTR (AF) and wt CFTR (wt) in BHK cells with, and without, prior growth in medium containing either 10% glycerol or 1 mM sodium 4-phenylbutyrate (4-PBA) for 24 hours monitored using a three stage ELISA (SEM; n=3). (B) Immunoblot showing the effect of the treatment in FIG. 3A on expression of ΔF508 and wt CFTR with, and without, glycerol treatment. (C) Densitometry of the immunoblot in FIG. 3B to determine the relative amounts of band B and C in the blot. (D) Confocal images taken of BHK cells expressing either ΔF508-CFTR (AF) and wt CFTR (wt) grown either at 37° C. or 27° C. in the presence of 10% glycerol. (E) Confocal microscopy to determine the level of permeabilization that occurred during the assay. BHK cells expressing ΔF508-CFTR were fixed and stained with wheat-germ agglutinin (WGA) to mark the cell surface. The WGA NON-PERM image shows the level of membrane staining with cells that were only fixed and stained. The WGA PERM image shows cells fixed and then permeabilized with 0.1% Triton X-100. The screen image shows the level of permeabilization of cells after they have undergone the screening process. (F) Histogram showing the range of fluorescence signals obtained in the HTS assay between the untreated ΔF508-CFTR and the wt CFTR or permeabilized ΔF508-CFTR BHK cells as compared to treatment for 24 hours with osmolite and/or temperature reduction (SEM; n=3).

FIG. 4: Properties of sildenafil as a CFTR corrector. (A) Structure and chemical name of sildenafil. (B) Ability of sildenafil to correct trafficking as monitored by immunoblotting. (C) Densitometry to quantify the amount of correction caused by sildenafil shown in 3B. (D) Iodide efflux assay to monitor the functionality of rescued ΔF508-CFTR at the plasma membrane in BHK cells after treatment with sildenafil (10 μM and 1 mM) for 24 hours prior to the assay.

FIG. 5: Differing regimes of fixation for cells affect the read out of the CFTR corrector screen assay. All fixations were performed at 4° C. The time of fixation allotted varied depending on the type of fixation with Methanol, Acetone and Methanol/Acetone all being carried out for 3-5 minutes; the Paraformaldehyde was used for 20 minutes and the fixation that included Glutaraldehyde was performed for 30 minutes. Success was judged by the range of difference between cells grown at 37° C. and 27° C. in particular the ΔF508 expressing cells with the larger the range the better. Using this criterion 2% Paraformaldehyde was considered the best.

FIG. 6: Test to identify if any already reported compound was suitable to be used as a positive control for the corrector assay screen. The increase in signal obtained for kifunesine, Sodium 4 Phenylbutyrate and the mix of curcumin and Sodium 4 Phenylbutyrate was deemed not sufficient to act as a positive control and instead post-fixation permeabilization using Triton X-100 was adopted. Concentrations used curcumin (40 μM); lactacystin (20 μM); geldanamycin (0.1 μg/ml); kifunesine (100 μM) and Sodium 4 Phenylbutyrate (1 mM), the combinations used the same concentrations and all were treated for 24 hours prior to fixation.

FIG. 7: Functional assay done in triplicate with two compounds. The solid line marks the negative control, the hatched line represents the positive control and the dotted line marks the test compound. The top compound, A (glafenine), was considered to test positive in each case, whereas the bottom three graphs demonstrate a compound (cyanocobalamin) that was not functional.

FIG. 8: Demonstration of the effect of the Prestwick 15 compounds on CFTR trafficking in BHK cells. Cells were cultured for 48 hours in the presence of 10 μM concentrations of the various compounds at 37° C. prior to lysing and immunoblotting for CFTR and tubulin. NC is the negative control and is the same cells treated with only Carrier compound (DMSO) The positive controls in the final two lanes are cells grown at 27° C. and the wild-type version of the CFTR expressed in BHK cells.

FIG. 9: Functional assay for the Prestwick 15 compounds. (A) BHK iodide efflux; and (B) CFBE iodide efflux.

FIG. 10: Results of corrector assays, functional assays and ex viva and in vivo murine experiments for the Prestwick 15 compounds.

FIG. 11: Functional demonstration of synergy. (A) Telenzepine and AMP; and (B) Iodide efflux of pramoxine and lactobionic acid.

FIG. 12: Ex vivo rescue of ΔF508-CFTR in mouse ileum by corrector Cor 325, sildenafil analogue KM60 and glafenine. (A) Representative short-circuit current (I_{sc}) response to 10 μM forskolin and 10 μM CFTRinh-172 using ileum from wild-type CFTR mice. (B)) Representative trace of the control short-circuit current (I_{sc}) response to 10 μM forskolin (Fsk) and 50 μM genistein (Gst) using ileum from ΔF508-CFTR mice that had been incubated ex vivo with vehicle alone (0.1% DMSO). Stimulation of electrogenic Na⁺-glucose co-transport by apical 10 mM glucose (apical solution normally contained mannitol instead of glucose) confirmed tissue viability. (C) Dependence of functional rescue on the concentration of the corrector Cor325. BHK cells were treated with Cor325 for 24 h prior to iodide efflux measurements (n=8 for each concentration). (D) Same as in (C), but using KM60, a sildenafil analogue, instead of Cor325. (E) Same as in (C), but using glafenine, instead of Cor325. (F) Bar graph showing responses to forskolin+genistein after ex vivo treatment (n=5 for each) with Cor325 and other drugs followed by Isc studies of freshly isolated ileum. Data are presented as mean±SEM and compared to the ΔF508-CFTR control. **p<0.01, ***p<0.001.

FIG. 13: Identification of glafenine as a ΔF508-CFTR corrector by high-throughput screening. (A) Schematic of the high-throughput screening process and validation of hit compounds used in this study. (B) Chemical structure of glafenine hydrochloride. (C) Effect of glafenine hydrochloride on the surface expression of ΔF508-CFTR. BHK cells expressing ΔF508-CFTR were pre-treated for 24 h with 0.1% DMSO (ΔF508, n=4), with 10 μM glafenine (glafenine, n=4), with 10 μM VRT325 (VRT325, n=4) or incubated at low temperature (29° C., n=4) prior to monitoring the surface expression in the high-throughput assay. A representative BHK cell line expressing wild-type CFTR (wt, n=4) are also shown for comparison. Data are presented as mean±SEM and compared to the control (***p<0.001). (D) Traces showing iodide influx in HEK293 cells that co-express ΔF508-CFTR and a halide-sensitive YFP. Cells were pre-treated for 24 h with 0.1% DMSO (ΔF508, n=3), 10 μM of glafenine (glafenine, n=3), 10 μM of VRT325 (VRT325, n=3) or low temperature (29° C., n=3). Correction of ΔF508-CFTR function was assayed in a plate reader as quenching of YFP fluorescence by iodide in the presence of 25 μM forskolin, 45 μM IBMX and 50 μM genistein.

FIG. 14: Effect of glafenine on the surface expression of ΔF508-CFTR. (A) Immunoblot showing ΔF508-CFTR in lysates of BHK cells treated with 10 μM glafenine for 24 h. Control ΔF508-CFTR cells were treated with vehicle alone (0.1% DMSO; negative control) or incubated at 29° C. for 24 h (positive control). BHK cells expressing the wild type-CFTR (wt) are also shown for comparison. Band C corresponds to mature, complex-glycosylated CFTR and band B to core-glycosylated CFTR. (B) Densitometry of three independent immunoblots monitoring the relative amounts of band C and band B. The relative percentage band intensity is the fraction of each CFTR glycoform (band B or band C) relative to the total density (i.e. band B+band C) in each lane.

FIG. 15: Functional rescue of ΔF508-CFTR by glafenine in BHK and CFBE41o⁻. (A) Iodide efflux assay of corrected ΔF508-CFTR at the plasma membrane in BHK cells after treatment with 10 μM glafenine (n=16) for 24 h. Stimulation was evoked by 10 μM forskolin (Fsk)+50 μM genistein (Gst). Control cells received vehicle alone (0.1% DMSO, n=32). (B) Histogram compares the stimulation of iodide efflux mediated by ΔF508-CFTR induced by glafenine (glafenine; n=16) negative control cells treated with vehicle alone (0.1% DMSO, n=32), and several positive controls; corrector cor4a (cor4a; n=14,), VRT325 (VRT325; n=32), and low temperature incubation (29° C.; n=16). The iodide efflux shown is the largest peak value measured after subtraction of the basal rate prior to stimulation. Data are presented as the mean±SEM. Significance compared to vehicle alone was determined using a t-test. ***p<0.001. (C) Dependence of functional rescue on the concentration of glafenine. BHK cells were treated with glafenine for 24 h prior to iodide efflux measurements (n=8 for each concentration). Results are also shown for control cells receiving vehicle alone (0.1% DMSO, n=22), and cells treated for 24 h with the corrector cor4a (10 μM; n=24) or low temperature for 24 h (29° C.; n=24). (D) Same as in (B), but using CFBE41o⁻ airway epithelial cell line expressing ΔF508-CFTR.

FIG. 16: Rescue of ΔF508-CFTR in human bronchial epithelia (CFBE41o⁻). (A-C) Representative traces of the short-circuit current (I_{sc}) responses to 10 μM forskolin, 50 μM genistein and 10 μM CFTRinh-172 after 24 hour exposure of CFBE41o⁻ cells to (A) 0.1% DMSO; (B) 10 μM glafenine or (C) 29° C. for 24 h. (D) Histogram showing the change in I_{sc} (ΔI_{sc}) after addition of forskolin+genistein, defined as the difference between the sustained phase of the current response and the baseline immediately before stimulation. The stimulation of I_{sc} after treatment with glafenine (ΔI_{sc}) was compared with those after incubation with 10 μM VRT325 or low temperature (29° C.) for 24 h. Also shown are CFBE41o⁻ cells expressing wild-type CFTR (wt). Data are presented as the mean±SEM (n=8 for control, n=9 for glafenine, n=5 for VRT325, n=5 for 29° C. and n=14 for wt) and compared to the DMSO control. Difference significant from control *p<0.05, ***p<0.001.

FIG. 17: Ex vivo and in vivo rescue of ΔF508-CFTR in mouse ileum by glafenine. (A) Representative trace of the control short-circuit current (I_{sc}) response to 10 μM forskolin (Fsk) and 50 μM genistein (Gst) using ileum from ΔF508-CFTR mice that had been incubated ex vivo with vehicle alone (0.1% DMSO). Stimulation of electrogenic Na⁺-glucose co-transport by apical 10 mM glucose (apical solution normally contained mannitol instead of glucose) confirmed tissue viability. (B) Representative short-circuit current (I_{sc}) response to 10 μM forskolin, 50 μM genistein and 10 μM CFTRinh-172 using ileum from wild-type CFTR mice. (C) Rescue of the I_{sc} response to forskolin/genistein in ileum from ΔF508-CFTR mice after ex vivo incubation with glafenine (20 μM for 5-6 h). (D) Bar graph showing responses to forskolin+genistein after ex vivo treatment (black bar; n=5 for each) or in vivo treatment with glafenine followed by Isc studies of freshly isolated ileum (grey bar; n=4 for each). For in vivo experiments, mice were fed with saline containing glafenine (50 mg/kg) or vehicle alone (0.1% DMSO) by gavage for 2 days, once per day, and the ileum was dissected and immediately used to measure short-circuit current. Data are presented as mean±SEM and compared to the ΔF508-CFTR control. **p<0.01, ***p<0.001.

FIG. 18: In vivo stimulation of salivary secretion by glafenine in CF mice. (A) The average rates of salivary secretion by wild-type mice (wt; n=5), CF mice (ΔF508; n=5) or CF mice after being fed once per day with saline containing 50 mg/kg glafenine by gavage for 2 days, (ΔF508+glafenine; n=5) (B). Total salivary secretion for wild-type mice (wt; n=5), CF mice (ΔF508; n=5) or CF mice fed with saline containing 50 mg/kg glafenine by gavage fort days, once per day (ΔF508+glafenine; n=5). Data are presented as mean±SEM and compared to the ΔF508 control. **p<0.01, ***p<0.001.

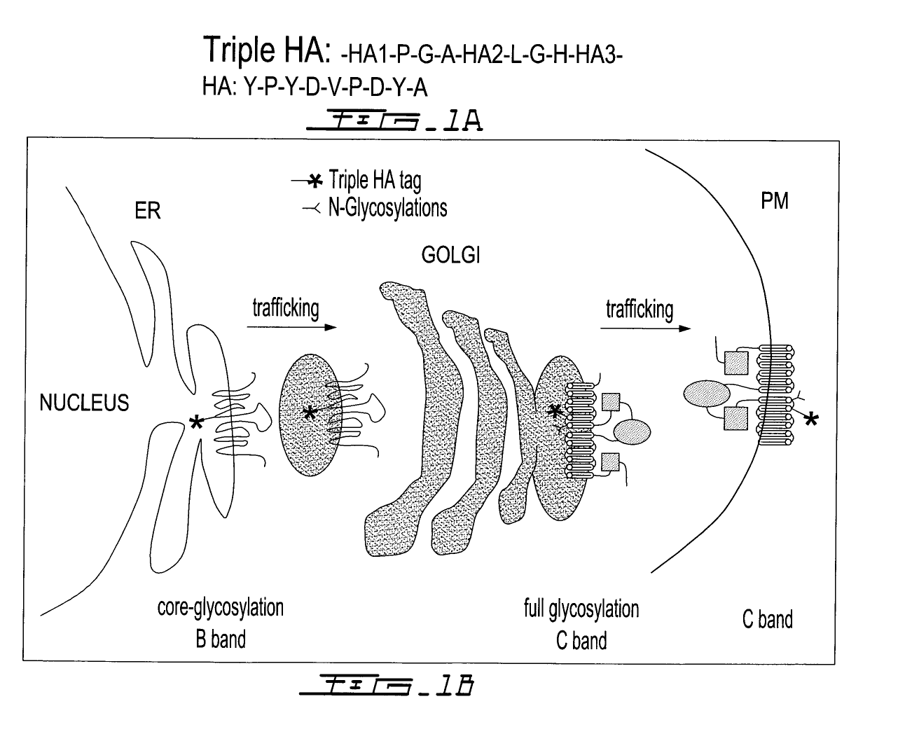

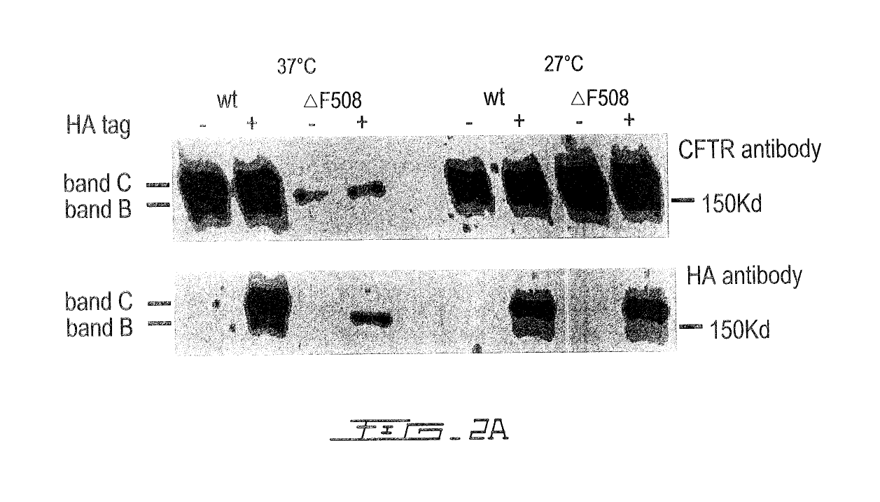

### DETAILED DESCRIPTION OF THE INVENTION

### Assay Development

Cells were screened for CFTR surface expression using a three stage ELISA system (Pierce Inc., USA). Briefly, BHK cells expressing 3HA-tagged ΔF508-CFTR were seeded in 96-well plates (Corning, USA) at 30,000 cells per well and incubated for 24 h at 37° C. Each well was treated with a test compound for 24 h, then cells were fixed in 4% paraformaldehyde solution and washed with PBS containing 0.1% bovine serum albumin and 0.05% tween-20. Cells were blocked for 1 h in solution containing 3% normal horse serum in PBS at room temperature. This solution was replaced with one containing primary antibody (mouse monoclonal anti-HA antibody, dilution 1:500, Sigma) for 2 hours at room temperature, then the wells were rinsed three times and tapped dry. Biotinylated anti-mouse secondary antibody was added (1:200 dilution, Pierce Inc., USA) in PBS containing 1.5% normal horse serum and incubated for 45 minutes at room temperature. Wells were washed four times, then exposed to tetravalent avidin peroxidase conjugate for 30 minutes at room temperature. After washing four more times, the cells were exposed to 3,3′,5,5′ tetramethylbenzidine (Pierce Inc., USA) for 15 min, sulphuric acid (2M) was added, and absorbance was measured at 450 nm (Power-Wavex Bio-tek Instruments).

### HTS Protocol

BHK cells expressing 3HA-tagged ΔF508-CFTR between passages 5-8 were seeded in 96-well plates (Corning half area, black-sided, clear bottom) at 15,000 cells per well and incubated with culture medium for 24 h at 37° C. Each well was then treated with a different test compound (80 compounds per plate) for 24 h at 20 μM final concentration. The remaining 16 wells on each plate were used for control conditions. Compounds were dissolved in DMSO which had no effect on trafficking when added at the same concentrations (data not shown). Cells were fixed in a 4% paraformaldehyde solution, washed with PBS, and blocked with PBS containing 5% fetal bovine serum (FBS) for 1 hat 4° C.

Blocking solution was replaced with 15 μl of primary antibody solution containing 1% FBS and mouse monoclonal anti-HA antibody (1:150 dilution, Sigma) in PBS. The plates were sealed and left at 4° C. overnight. After three washes with 100 μl PBS, cells were incubated for 1 h with 15 μl of secondary antibody solution containing 1% FBS and anti-mouse IgG conjugated with FITC (1:100 dilution, Sigma) in PBS. Cells were again washed three times with 100 μl of PBS and analyzed in a plate reader (Analyst™ HT96.384, Biosystems; 488 nm excitation/510 nm emission). Hits were defined as those compounds giving fluorescence at least three standard deviations higher than untreated controls. The mean fluorescence of four untreated wells was used as the background signal when calculating deviations of the 80 compound-treated wells. Hits were then cherry-picked into reservoir plates and re-tested in duplicate using the same assay. Compounds that consistently give signals that were three standard deviations above untreated controls and were not intrinsically fluorescent were considered validated and studied further.

### Preparation of Stable Cell Lines Expressing Tagged Wild Type and ΔF508-CFTR

CFTR tolerates insertion of tags into the fourth external loop therefore three haemaglutinin-epitope tags (YPYDVPDYA) were inserted in tandem after amino acid 901 in both wild-type and ΔF508-CFTR.¹⁰ Briefly, four primers covering sequence between an upstream Hpa1 restriction site at 2460 bp and a downstream Pml1 site at 3720 bp were used. PCR was used to synthesize a fragment containing three HA epitopes separated by amino acid linkers (HA1-P-G-A-HA2-L-G-H-HA3), which was then ligated into full length pNUT-CFTR linearized using Hpa1 and Pml1.

Cell lines expressing the tagged constructs were prepared as follows. Briefly, BHK cells were seeded at a density of 200,000 per well in a 6-well plate (Fisher) and allowed to grow to approximately 80% confluence. They were transfected using the lipophilic agent Lipofectamine Plus (Invitrogen) and 2 μg of pNUT-CFTR DNA that was replaced with fresh medium after three hours and transferred into 16 cm diameter dishes (Becton Dickinson) after 24 hours. Transfectants were selected in 500 μM methotrexate and single colonies were transferred to six well plates and tested for CFTR expression using both Western blot analysis and CFTR channel function by iodide efflux assay. The cystic fibrosis airway epithelial cell line CFBE41o⁻ (ΔF508/ΔF508) which was developed by Dr. D. Gruenert and colleagues,¹¹ and transduced with wild-type or ΔF508-CFTR using the TranzVector lentivirus system,¹² was generously provided by J. P. Clancy and cultured as described previously.¹³

### Western Blots

Cell lysates were quantitated by Bradford assay (BioRad) and separated by SDS-PAGE (6% polyacrylamide gels) and analyzed by Western blotting. Western blots were blocked using 5% skimmed milk in PBS, then probed overnight at 4° C. with a primary anti-CFTR antibody at a dilution of 1:1000 monoclonal mouse antibody (clone M3A7, Chemicon). The blots were washed four times in PBS before the addition of the secondary HRP-conjugated anti-mouse antibody, at a dilution of 1:5000 (Amersham) for one hour at room temperature. The blots were washed five times in PBS and probed for chemiluminescence (Pierce). All samples were run with equal protein loading as determined using the Bradford assay (Biorad). Densitometry of the immunoblots was performed using the ImageJ program (National Institutes of Health).

### Immunofluorescence

Cells were seeded onto 1 cm diameter glass coverslips (5000 per coverslip) and incubated overnight, then treated with compound and fixed in 4% paraformaldehyde. After fixation cells were blocked using 5% FBS in PBS for 1 hour at 4° C. The coverslips were then washed in PBS and incubated with primary antibody solution (1% FBS in PBS with 1:200 dilution mouse anti-CFTR antibody (clone M3A7, Chemicon) for 2 hours at room temperature (0.05% Tween-20 was added to the blocking solution when staining of intracellular CFTR was required). Coverslips were washed four times in PBS and probed with secondary antibody solution (1% FBS in PBS plus goat anti-mouse Alexa 568 conjugated antibody at 1:1000 dilution) for 1 h at room temperature in the dark. The cells were then washed three times with PBS. The coverslips were then mounted on slides using an antifade mounting solution (Permamount) for confocal microscopy.

### Iodide Efflux Assay

Experiments were performed by hand or with a robotic liquid handling system (BioRobot 8000, Qiagen, USA) using Qiagen 4.1 software. Cells were cultured in 24-well plates until they reached confluence in order to perform parallel experiments and comparison analysis. After treatment (or not) with a test compound, medium in each well was replaced with 1 ml of iodide loading buffer (in mM: 136 NaI, 3 KNO₃, 2 Ca(NO₃)₂, 11 glucose and 20 Hepes pH 7.4) for 1 hour at 37° C. to permit the I′ to reach equilibrium. At the beginning of each experiment, the loading buffer was removed by aspiration and cells were washed eight times with efflux buffer (same as loading buffer except that NaI was replaced with 136 mM NaNO₃) to remove extracellular I⁻ in each well. The loss of intracellular I⁻ was determined by removing the medium and replacing it with fresh efflux buffer every 1 min for up to 11 min. The first four aliquots were recovered at 1-minute intervals in an empty 24-well plate and used to establish a stable baseline in efflux buffer alone. A stimulation buffer (efflux buffer containing 50 μM genistein+10 μM forskolin) was then added and sampling continued with replacement by stimulation buffer. The iodide concentration of each aliquot was determined using an iodide-sensitive electrode (Orion Research Inc., Boston, Mass., USA or Ecomet) and converted to iodide content (i.e. the amount of iodide released during the 1 min interval). Curves were constructed by plotting concentration versus time. Data are presented as means±SEM.

### Insertion of the Triple Hemagglutinin (3HA) Tag has Minimal Effects on the Processing and Functionality of Wild-Type (wt) or ΔF508-CFTR

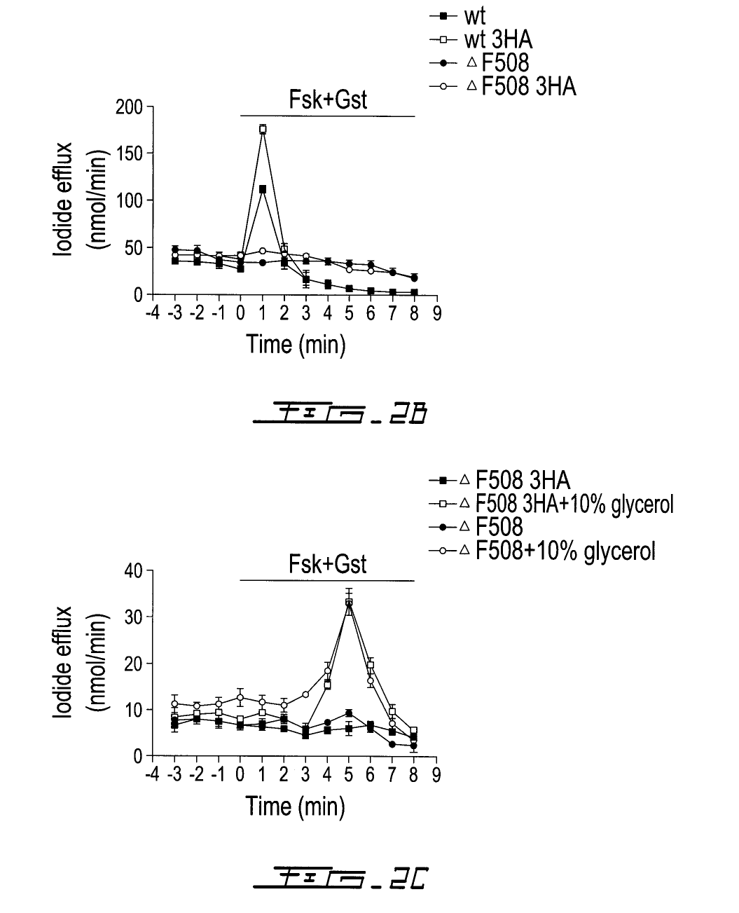

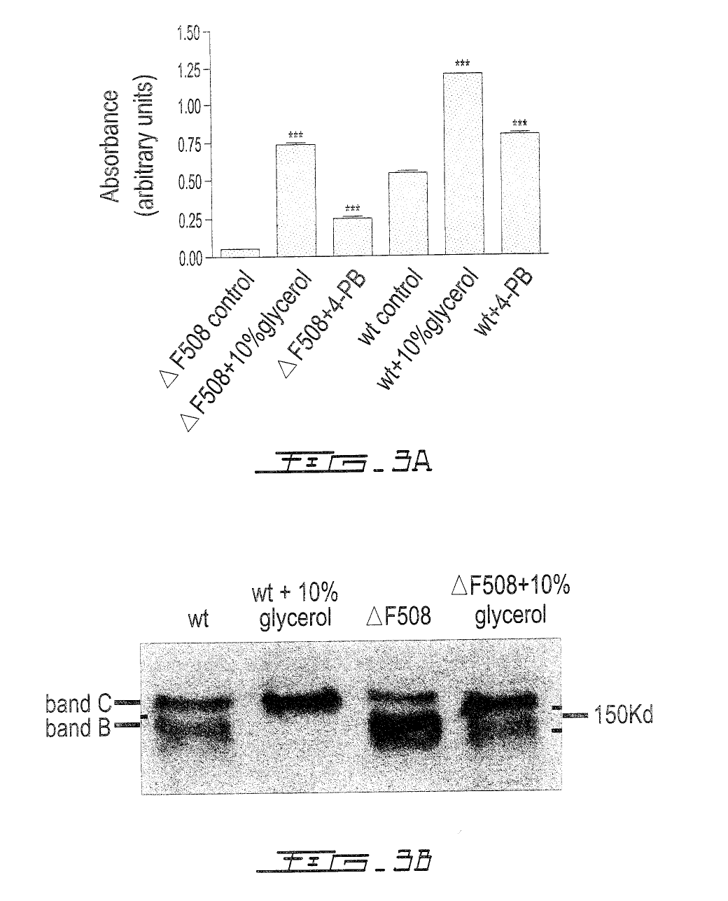

The effect of inserting an extracellular 3HA tag on CFTR processing was assessed by immunoblotting. Accumulation of a mature, complex-glycosylated CFTR (band C) form was used as evidence of correct trafficking. BHK cells were incubated at 37° C. or 27° C. in a 6-well plate for 48 hours (FIG. 2A). Insertion of the 3HA tag did not alter the amount of band C noticeably. As reported previously, the band C of CFTR was not observed in ΔF508 expressing cells growing at 37° C., however, band C did appear if cells were cultured at 27° C.⁷ The conclusion drawn here is that the 3HA-tag has little effect on the synthesis and trafficking of wild-type or mutant forms of CFTR, and that both mature and immature forms are readily detected using an anti-HA antibody (FIG. 2A).

To determine if the 3HA tag affects ion channel function, cAMP-stimulated halide permeability was analyzed using an iodide efflux assay (FIG. 2B). Cells expressing either tagged or untagged wild-type CFTR mediated a similar iodide efflux upon cAMP stimulation whereas no cAMP mediated iodide efflux response was observed from the ΔF508-CFTR cells with or without the 3HA tag (FIG. 2B) or from parental BHK cells lacking CFTR (data not shown).

The 3HA tag did not disrupt the activity of rescued ΔF508-CFTR at the cell surface (FIG. 2C). Low basal efflux was observed when a tagged version of ΔF508-CFTR was stably expressed in BHK cells under normal conditions, as expected when most of the protein is misfolded and retained in the endoplasmic reticulum. However, when those cells were grown at 27° C. with 10% glycerol in the medium, a cAMP stimulated efflux was readily detected, showing that the 3HA-tag did not inhibit channel function. The rescued iodide efflux was smaller and delayed compared to the iodide efflux obtained with cells expressing wt CFTR but was similar to that seen for the untagged ΔF508-CFTR control cells (data not shown).

### Optimization and Characterization of the Screening Assay for ΔF508-CFTR Trafficking Correctors

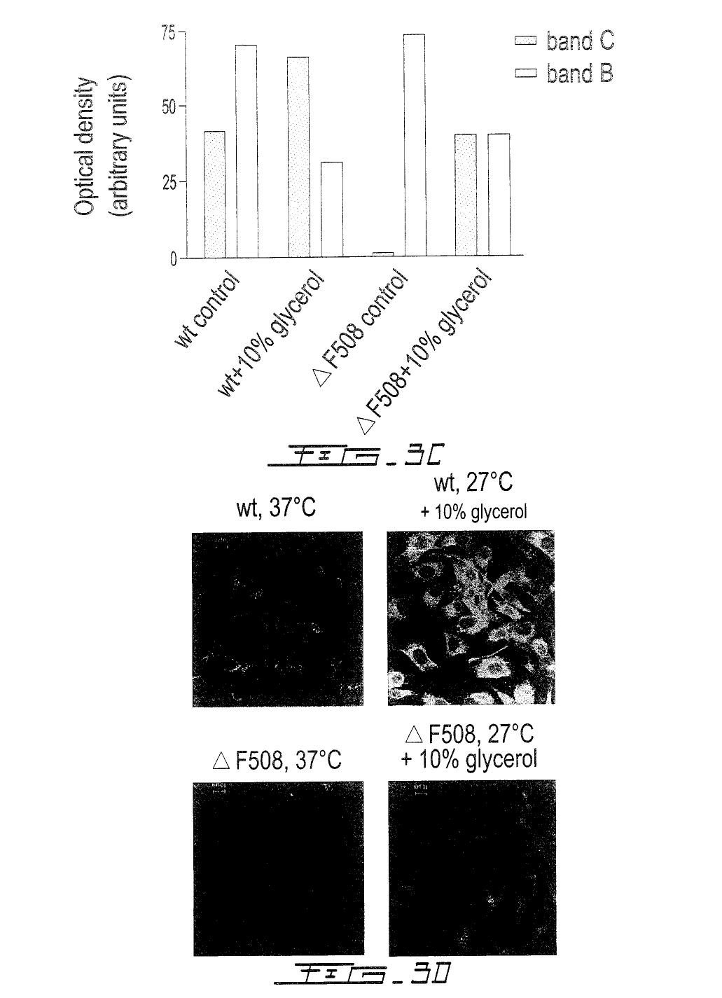

To determine if cell surface expression of CFTR could be quantified in a high-throughput screen using this assay, cells expressing tagged versions of wt or ΔF508-CFTR were seeded in 96-well plates, treated with 10% glycerol and monitored as light absorbance using an enzyme-linked assay system. As expected, untreated ΔF508-CFTR cells had low absorbance since they expressed little CFTR on their cell surface under control conditions (FIG. 3A). By contrast, cells expressing wt CFTR had a signal that was 10-fold higher. Treating cells with chemical chaperones such as glycerol and sodium 4-phenylbutyrate increased the plasma membrane signal, but this effect on surface expression was much stronger with cells expressing the mutant ΔF508 than with cells expressing wild-type CFTR, where glycerol treatment caused only a 2.5-fold increase (FIG. 3A).

Immunoblotting confirmed that the magnitude of the fluorescence signal was related to the extent of ΔF508-CFTR trafficking corrections (FIG. 3B). The band C forms of wt and ΔF508-CFTR were both increased by treatment with 10% glycerol according to densitometry, but again the increase was much larger for ΔF508-CFTR (37 fold) than for wt CFTR (1.5 fold) (FIGS. 3B and 3C).

Differences in CFTR surface expression were visualized by confocal microscopy (FIG. 3D). Little fluorescence was measured on the ΔF508-CFTR cells maintained at 37° C. As shown in FIG. 3D, the ΔF508-CFTR expression was markedly increased upon treatment with 10% glycerol and incubation at 27° C.

A range of fixation protocols were tested to minimize cell permeabilization, since this would lead to false-positives in the screen. Treating cells with 4% paraformaldehyde in PBS for 20 minutes at 4° C. was found to be optimal (data not shown). Wheat Germ Agglutinin (WGA) staining of the plasma membrane was restricted to the surface in non-permeabilized cells, in marked contrast to the intracellular staining cells exposed to mild detergent (0.1% Triton X-100) (FIG. 3E). No internal staining was detected when assays were complete suggesting that handling and exposure to compounds did not cause significant permeabilization. Similarly, a variety of antibodies were tested to optimize the signal fluorescence the assay (data not shown).

Despite the strong fluorescence of cells after treatment with 10% glycerol, this chemical chaperone was not suitable as a positive control in the high-throughput assay. Several compounds reported previously to correct ΔF508-CFTR processing defect were tested for use as potential positive controls but with limited success (data not shown). Using this assay, control experiments confirm that significant changes were detectable (FIG. 3F). After treatment of the ΔF508-CFTR cells with 10% glycerol or low temperature respectively, a 2.7 and 2.3 fold fluorescence increase was detected in comparison with the untreated cells (FIG. 3F). An additive effect was observed when cells were incubated with both treatment. A 5 and 7.6 fold increase fluorescence signal was detected in untreated wild-type CFTR and permeabilized cells respectively as compared to the untreated control (FIG. 3F). Consequently, permeabilized cells were used as a positive control for fluorescence and hence total CFTR protein expression (FIG. 3F). This had the added advantage that fluorescence signals above the positive control indicated greatly increased CFTR expression allied to trafficking or, more likely, that the compound was intrinsically fluorescent.

Example 1: Screening Compounds from Microsource Discovery (MDS)

A total of 2000 diverse drug-like compounds were used in the screen from Microsource Discovery.

### Identification of ΔF508-CFTR Correctors and Characterization of Sildenafil as a Hit Compound

BHK cells expressing ΔF508-CFTR were incubated with test compounds (20 μM) for 24 hours at 37° C. in a 96 well format. Plasma membrane expression of ΔF508-CFTR was then assayed by immunofluorescence using a primary antibody directed against the inserted 3HA tag and a secondary antibody conjugated with a fluorophore (FITC). Untreated cells probed with the same antibodies were used as a negative control, and cells exposed to 0.1% Tween-20 detergent (so that antibodies had access to intracellular CFTR) served as a positive control. In the primary screen, strong hits were initially identified as those compounds giving a cell fluorescence signal that was ≧3 standard deviations (SD) above untreated control wells. Medium and weak hits were defined as compounds giving a cell fluorescence signal that was between 2 and 3 or 1 and 2 standard deviations, respectively, above untreated control wells. The positive compounds were selected and retested in duplicate to obtain an N of 3. At the re-screening stage, the intrinsic fluorescence of each potential hit was also measured and the fluorescent ones were not considered further.

With this system, the 2000 compounds in the Microsource Discovery library of small molecules were screened and 16 strong hits were obtained (SD>3). These and 64 weaker hits were cherry picked and re-tested in duplicate. Of the 16 strong hits from the primary screen 13 were confirmed by re-testing, however, of these 8 proved to have intrinsic fluorescence and were discarded. Therefore the screen yielded 5 strong hits, 6 medium hits (2 SD<3) and 18 weak hits (1 SD<2) (Table 1). The screen confirmed six compounds that other CFTR assays have previously shown to influence CFTR, and also identified novel families of compounds. Of the 5 strong hits, one was chlorzoxazone, a compound that had already been identified as a possible CFTR activator.¹⁴ However, the other four dacthal, glycyrrhizic acid, chloramphenicol and carboplatin had not been reported and hence were novel correctors. Further, 24 other compounds (medium and weak hits) were found to give a consistently elevated signal in the assay. Of this group, six had previously been reported to affect CFTR trafficking function: sildenafil, daidzein, dyhydroepiandrosterone, 3,3,5-triiodothyronine, bromhexine and khivorin.¹⁵⁻¹⁸ Also, a further 4 are analogs of khivorin and dyhydroepiandrosterone; didectylkhivorin, 1-deacetoxy-1-oxo-3,7 dideactyl khivorin, 3-beta chloroandrostanone and epiandrosterone. The rest had not previously been identified as contributing to CFTR trafficking and may be regarded as novel primary hits identified by this assay (Table 1). To further validate the screen, a decision was made to test one of the hits, sildenafil.

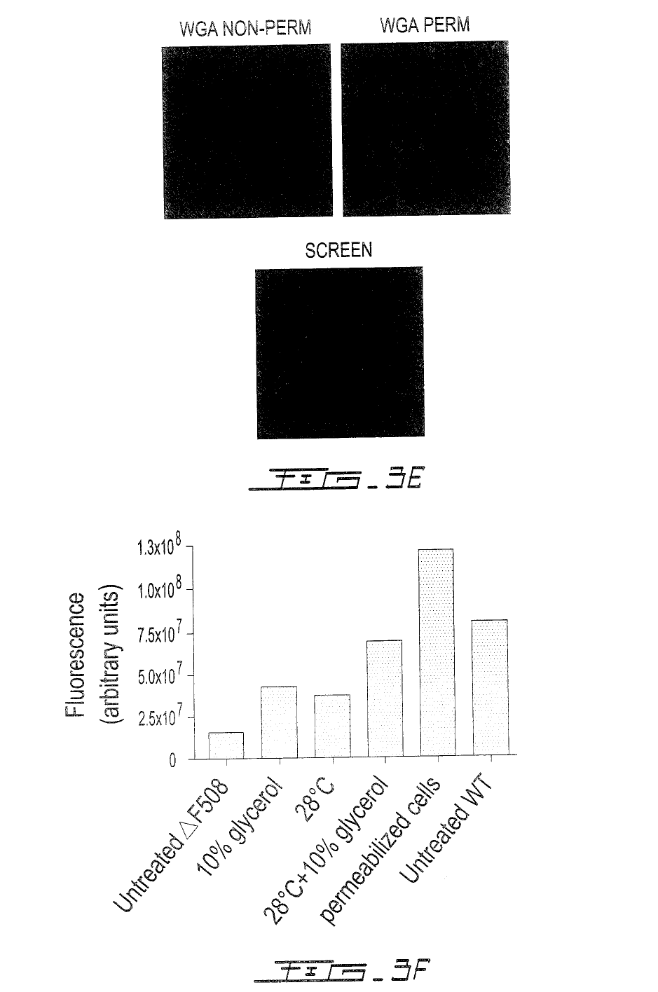

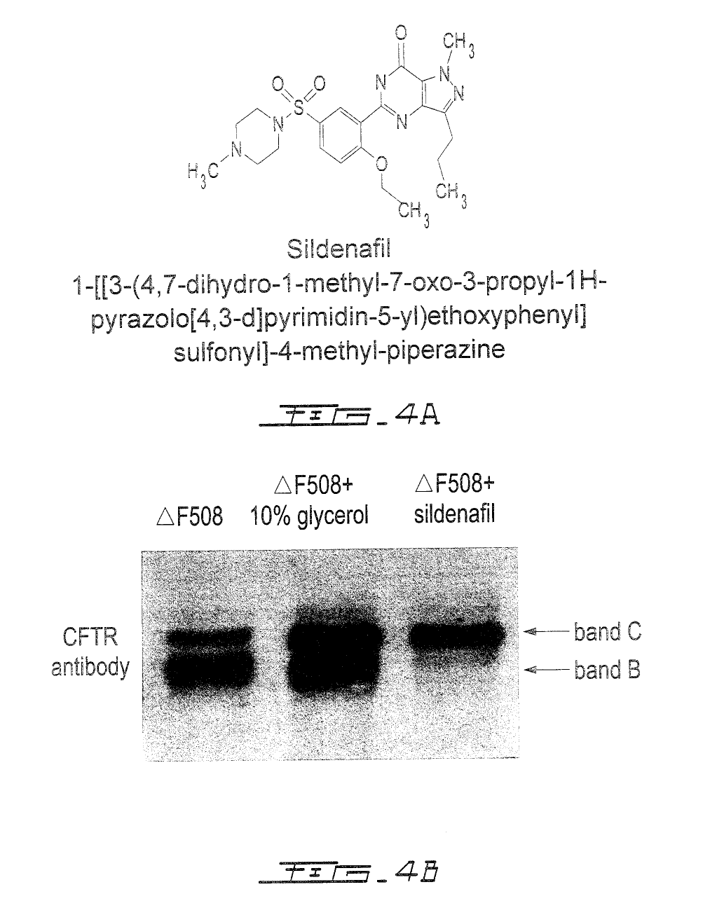

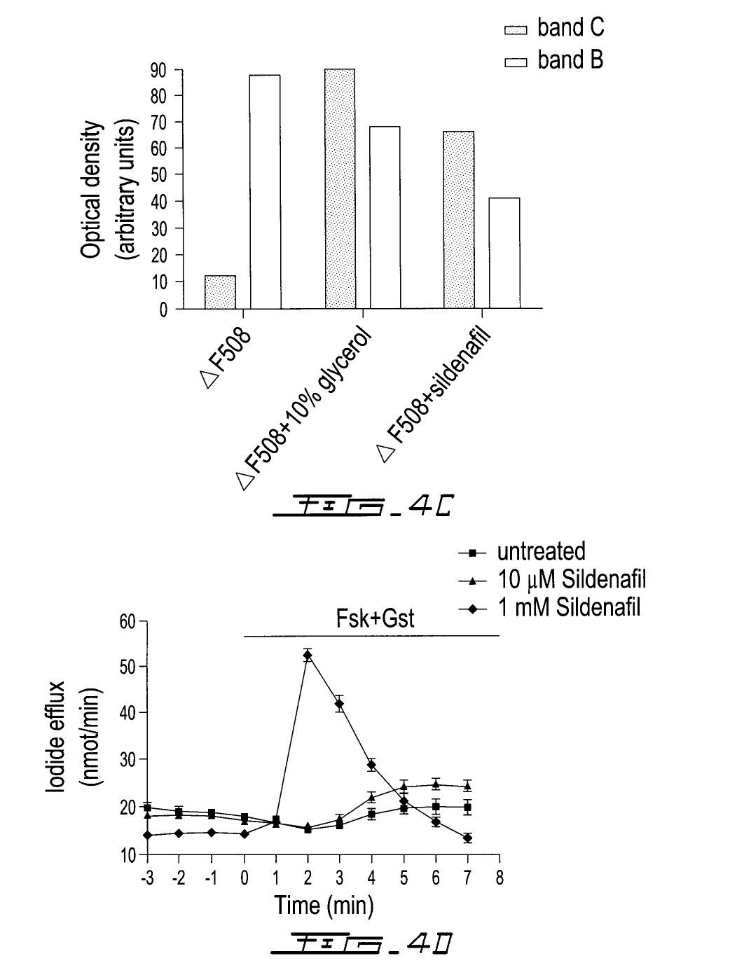

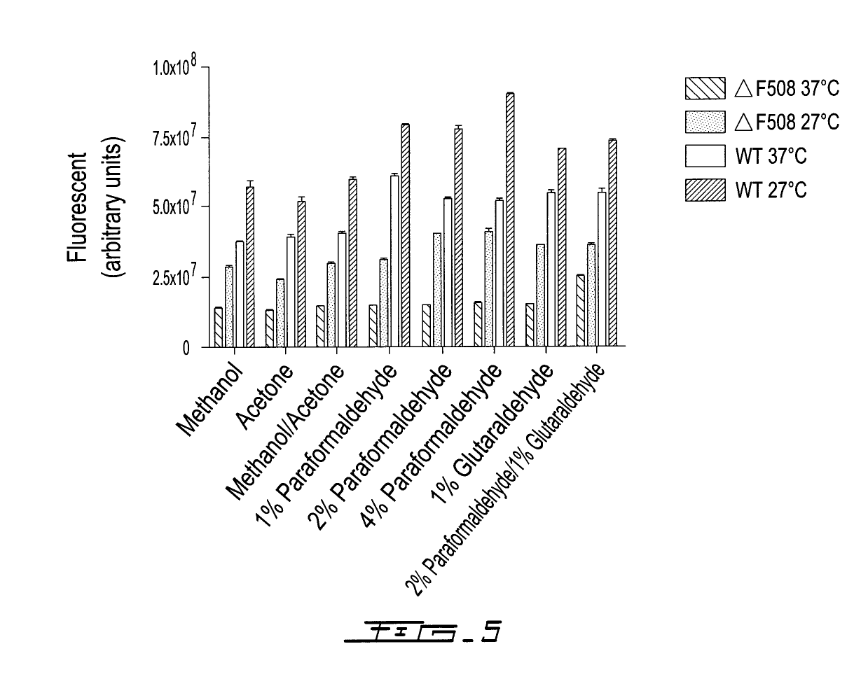

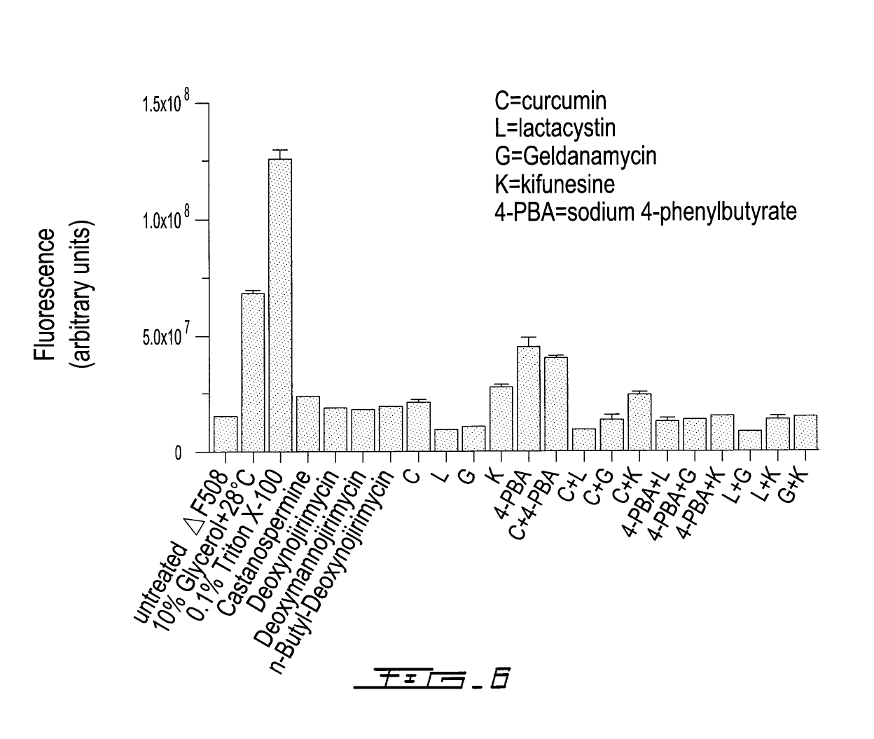

Sildenafil is a phosphodiesterase inhibitor (PDE-5) (FIG. 4A) that has been previously reported as a CFTR corrector at millimolar concentrations, however, the HTS assay revealed correction with only 20 μM, and partial correction of trafficking was confirmed by immunoblot analysis (FIG. 4B).¹⁸ This suggested that properly folded CFTR trafficked to the cell surface. After sildenafil treatment, ΔF508-CFTR cells showed a 30% increase in the intensity of the mature band C and a concomitant decrease in the immature band B (FIG. 4C).

Surprisingly, pretreatment with 10 μM sildenafil pretreatment did not restore a strong cAMP-stimulated iodide efflux responses, suggesting that little of the mature CFTR protein was functional at the cell surface (FIG. 4D). The inability to detect significant channel activation after pretreatment with 10 μM sildenafil was further confirmed using the CFBE41o⁻ airway epithelial cell line derived using tissue from a cystic fibrosis patient, which also gave a negative result when used in an iodide efflux assay (data not shown). However, if sildenafil was used at 1 mM for 24 hours prior to assaying for functional channels in BHK cells a strong cAMP-stimulated iodide efflux was detected (FIG. 4D).

To evaluate the screen and assay alone, the Z score and Z′ score, respectively, were calculated.¹⁹ For the MSD screen the Z score was 0.519 indicating considerable separation between scores for controls and active compounds. The Z′ score for the assay was 0.728, which demonstrates robust correction in the screening assay and suggests that it will be useful for identify correctors by HTS.

Example 2: Screening of Additional 1120 FDA Approved Drugs from the Prestwick Library

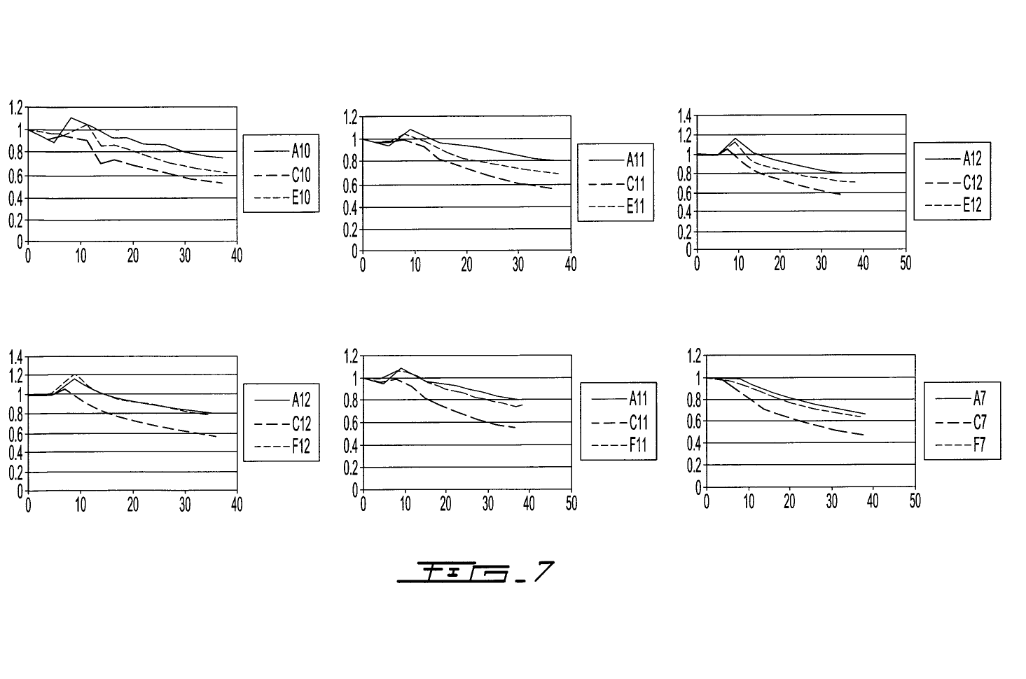

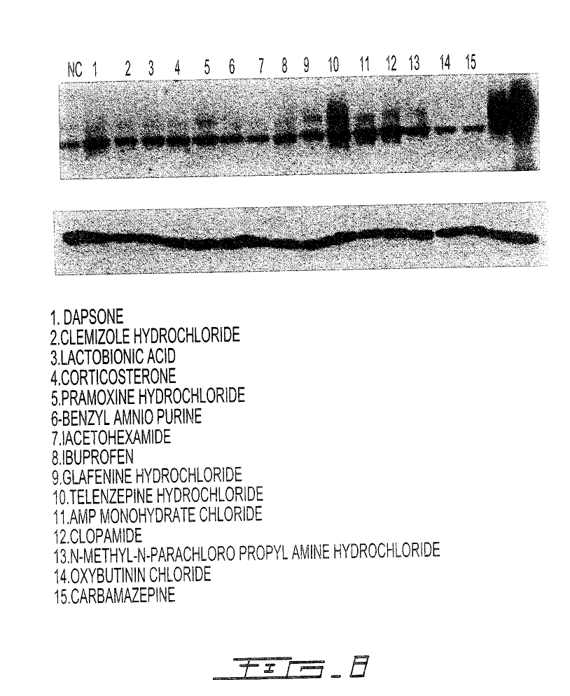

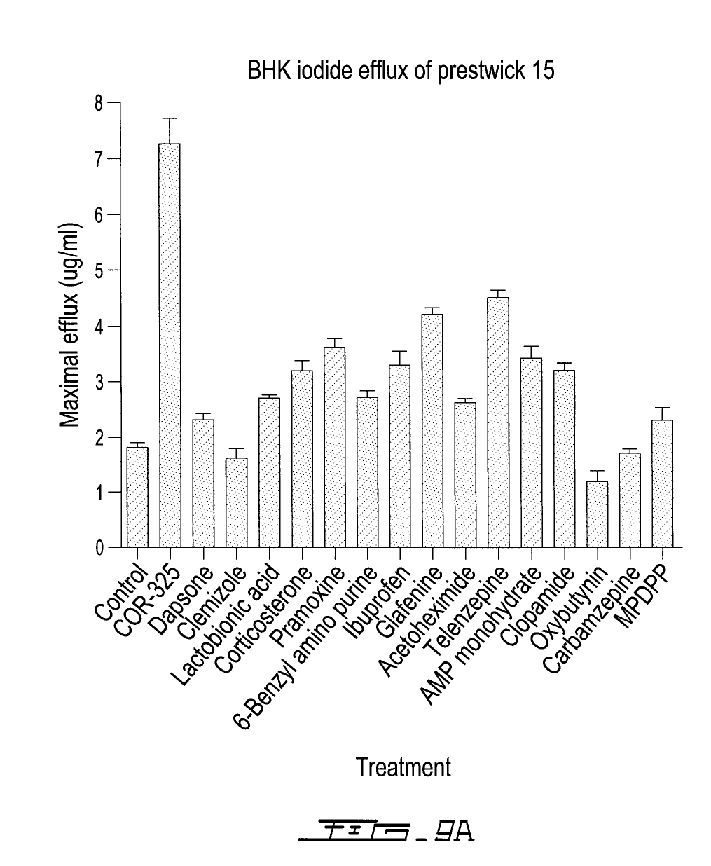

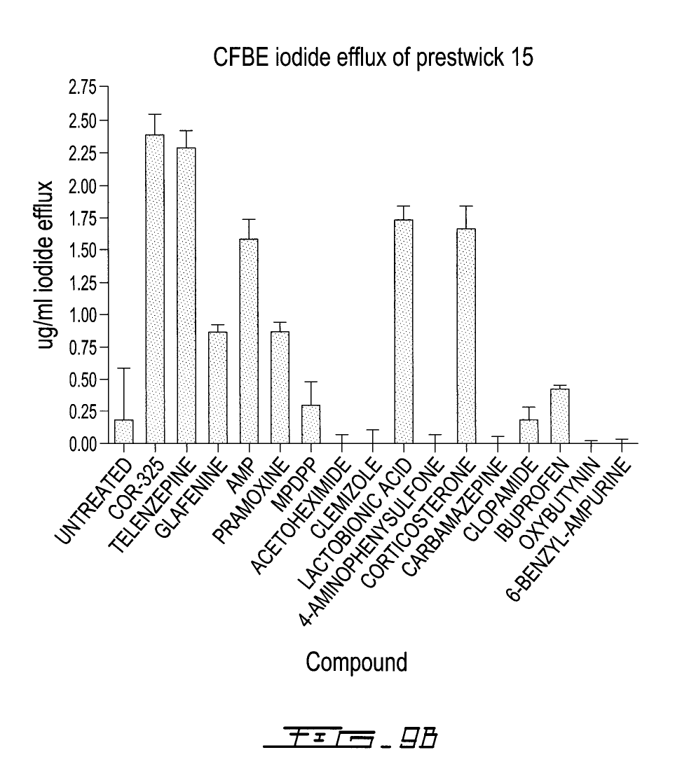

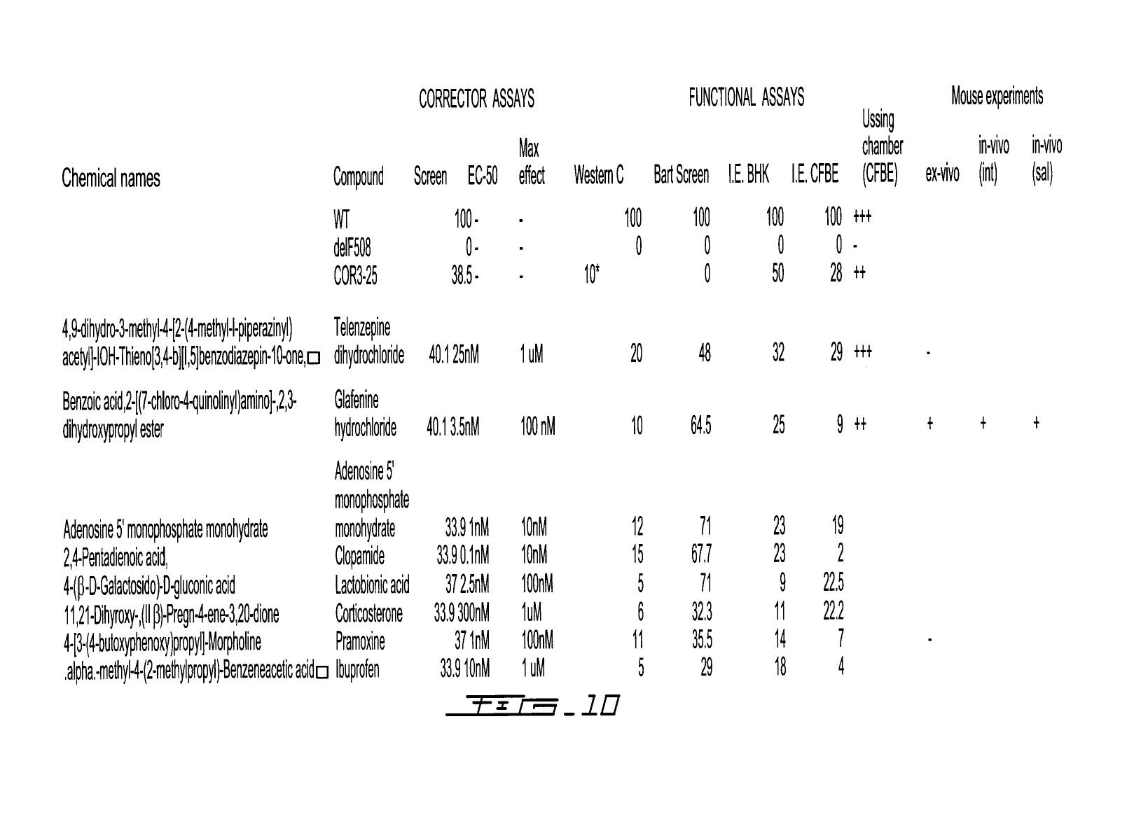

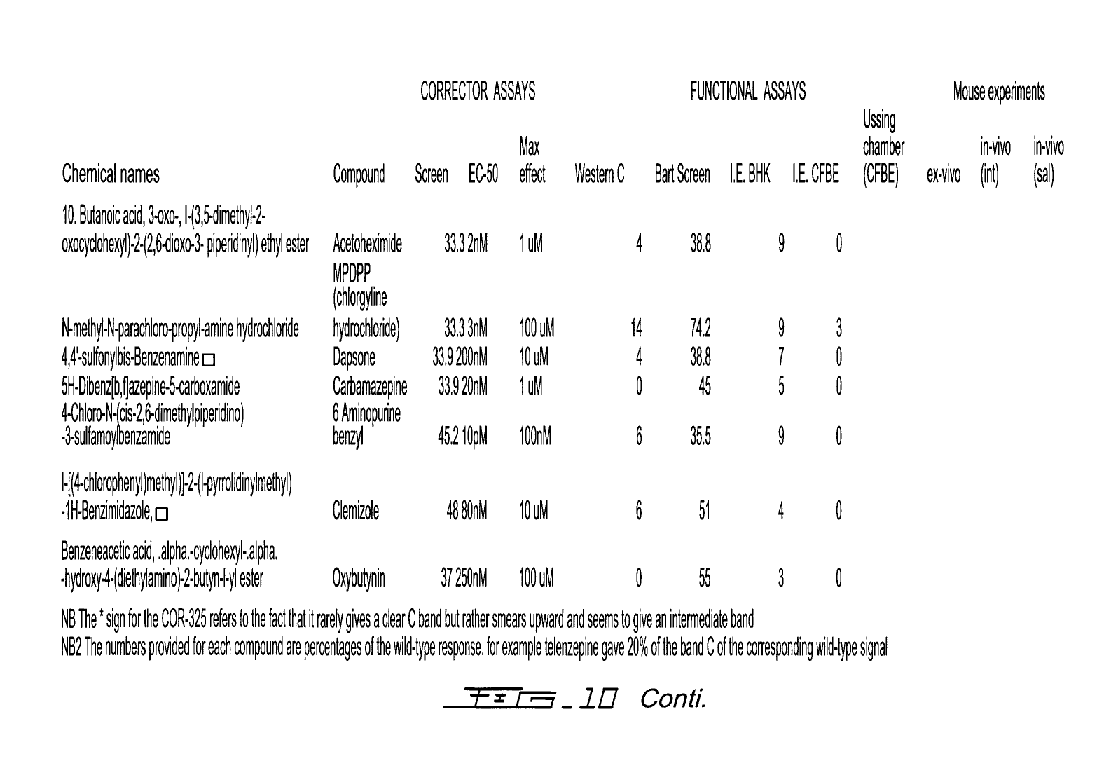

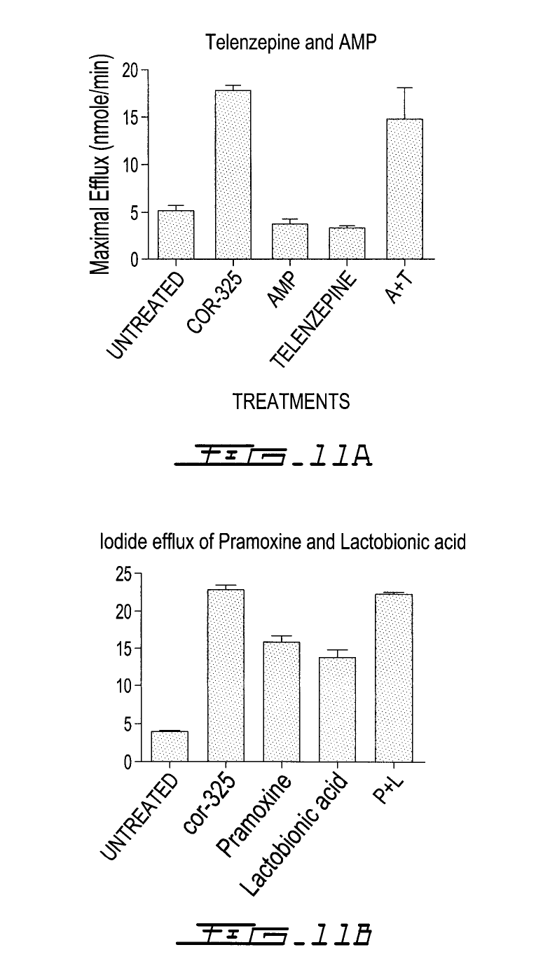

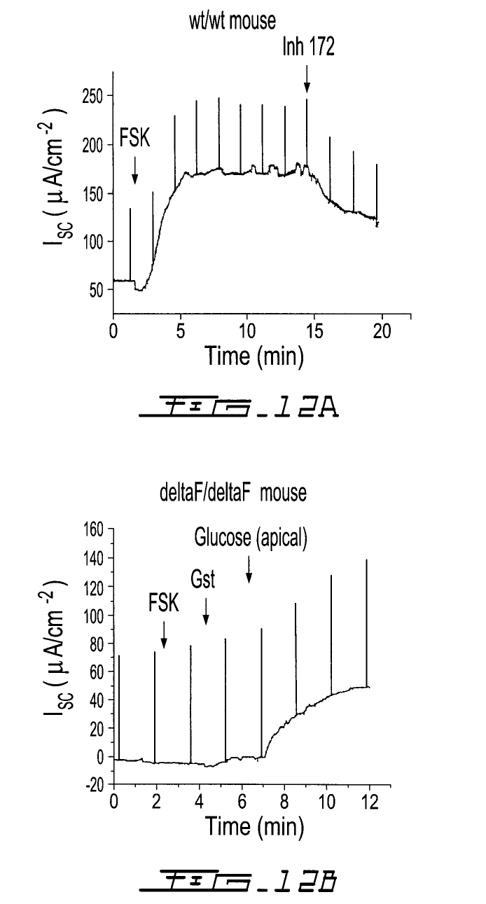

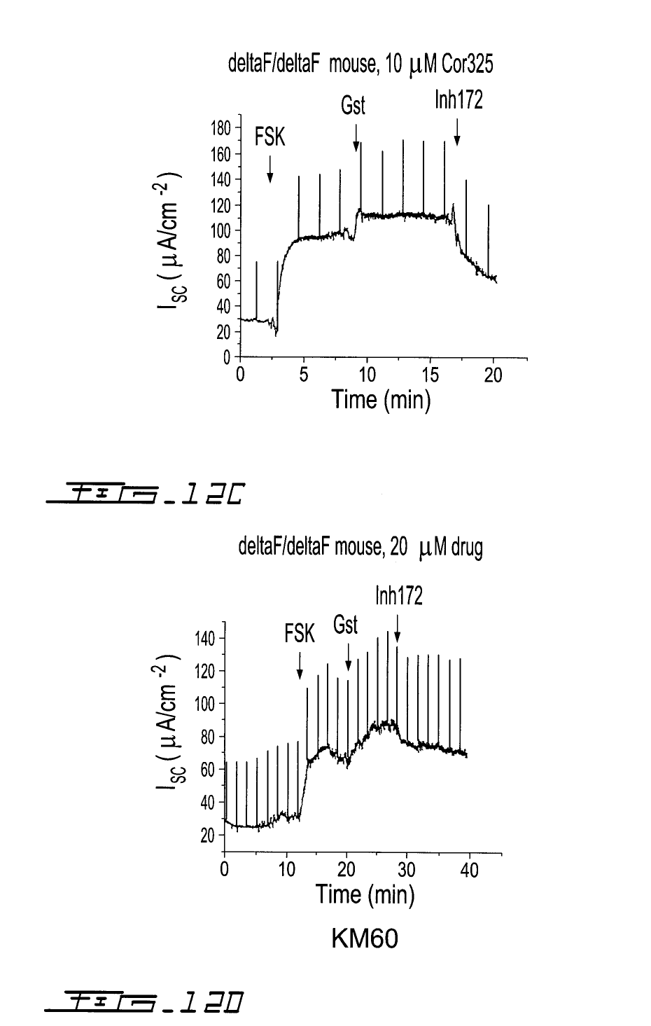

In addition to the MDS compounds, the 1120 Prestwick Library compounds were screened. (See FIGS. 7 to 12.) Of the compounds, one qualified as a “strong” hit, 3 as “medium” hits and 57 as “weak” hits. The Z score for the assay was 0.62. The top 50 hits were further tested in a counter screen in which the halide sensitive fluorescent compound (YFP) was expressed inside cells (Table 2). After incubation with the test compounds the ability of the cells to uptake iodide was measure by decreasing YFP fluorescence. In these cells this can only occur if functional CFTR is present at the cell surface (FIG. 7). The hits were also tested for the appearance of the Band C form of CFTR. This is the mature form of CFTR and occurs only if the protein has left the ER and entered the Golgi apparatus and hence is trafficking (FIG. 8). The functionality of the compounds was further studied by the use of Iodide efflux assays for each compound in both BHK and the more physiologically relevant CFBE cells. The results (FIG. 9) show that several of the compounds cause the appearance of function CFTR channels at the cell surface upon treatment for 24 hours. FIG. 10 is a table of results for the 15 hit compounds which summarizes many experiments. With the exceptions of the absolute values for the EC₅₀ and value for maximal effect all the results are given as percentages relative to wild-type response with wild-type scored as 100% and deltaF508 scored as 0%. FIG. 11 records the results when two compounds were tested together to see if they had any synergistic effect. The experiments carried out in Iodide efflux assays suggest that telenzepine and AMP do have a synergistic effect and that Pramoxine and Lactobionic acid have an effect that may be characterized as additive. The results for the best 15 compounds from the Prestwick Library, or “Prestwick 15”, are shown FIGS. 8, 9 and 10.

Protein folding and its subsequent trafficking are complex processes that are expected to have many potential sites of therapeutic intervention. Unlike previous work which assayed protein function as the end point,³ use was made of an approach in which the mutated protein of interest is tagged so that its trafficking to the surface can be monitored. This approach is less stringent but offers a more direct and general approach than measuring stimulation of chloride conductance, and can be used for any disease in which trafficking of a protein to the plasma membrane is abnormal. Once a corrector has been identified by HTS, characterization of its mode of action can then be used to gain insights into many cellular processes including protein translation, folding, golgi transport, glycosylation, transport to the plasma membrane and endosome recycling.

### Example 3: Correction of ΔF508-CFTR Trafficking Defect by the Bioavailable Compound Glafenine

### Materials and Methods

### HTS

Screening was performed using BHK cells that stably express F508-CFTR bearing three tandem haemagglutinin-epitope tags (3HA) and linker sequences in the fourth extracellular loop after amino acid 901 (Howard et al., 1995; Carlile et al., 2007; Robert et al., 2008). Rescue of the mutant by test compounds was monitored using a plate reader by measuring antibody binding to cells that had been fixed with paraformaldehyde (for details see Carlile et al., 2007). Screening was performed using the Prestwick Chemical Library of 1120 high-purity compounds.

### Immunoblot Analysis

Total protein was quantified in cell lysates using the Bradford assay (BioRad, Hercules, Calif.), separated using SDS-PAGE (6% polyacrylamide gels), and analyzed by Western blotting as described previously (Robert et al., 2007). Western blots were blocked with 5% skimmed milk in PBS and probed overnight at 4° C. with a monoclonal primary anti-CFTR antibody (clone M3A7, Chemicon, Temecula, Calif.) diluted 1:1000. The blots were washed four times in PBS before adding the secondary HRP-conjugated anti-mouse antibody at a dilution of 1:5000 (Amersham, Piscataway, N.J.) for one hour at room temperature, then washed again five times in PBS and visualized using chemiluminescence (Pierce, Rockford, Ill.). The relative intensity of each CFTR glycoform (band B or band C) was estimated by densitometry using the ImageJ software and reported as a percentage of the total CFTR in the same lane (i.e. B+C).

### YFP Fluorescence Assay

Strongly adhesive Human Epithelial Kidney cells stably expressing both the human macrophage scavenger receptor (HEK293 Griptite, Invitrogen) and F508del CFTR were plated in 96-well plates and transiently transfected with pcDNA3 plasmid encoding a halide sensitive variant of eYFP. After 24-48 h later cells were exposed to 10 μM test compound in triplicate and incubated for an additional 24 h. Cells were stimulated for 20 minutes with a in a buffer containing 25 μM forskolin, 45 μM IBMX and 50 μM genistein final concentration. The high content screening assay was performed using a Cellomics platform. Iodide was added robotically to a final concentration of 50 mM and the resulting decrease in fluorescence was measured. Images were taken at time 0 and stored for subsequent use when calculating a mask so that only those cells that express YFP at time 0 are measured. The quenching was detected in 15 images taken over the course of an experiment lasting 40 seconds. Each test compound was compared to the following two controls: a negative control without drugs to assess photobleaching that may occur when the same field of view is repeatedly imaged, and a positive control with cells treated with a known potentiator. Results were generated from 150-300 cells per well.

### Halide Flux Assay

Iodide effluxes were performed using a robotic liquid handling system (BioRobot 8000, Qiagen, Valencia, Calif.) and Qiagen 4.1 software. Cells were cultured to confluence in 24-well plates. After treatment (or not) with a test compound, the medium in each well was replaced with 1 ml of iodide loading buffer: 136 mM NaI, 3 mM KNO₃, 2 mM Ca(NO₃)₂, 11 mM glucose, 20 mM Hepes, pH 7.4 with NaOH) and incubated for 1 h at 37° C. At the beginning of each experiment, the loading buffer was removed by aspiration and cells were washed eight times with 300 μl efflux buffer (same as loading buffer except that NaI was replaced with 136 mM NaNO₃) to remove extracellular I⁻. Efflux was measured by replacing the medium with 300 μl fresh efflux buffer at 1 min intervals for up to 11 min. The first four aliquots were used to establish a stable baseline, then buffer containing 10 μM forskolin+50 μM genistein was used to stimulate CFTR activity. Iodide concentration was measured in each aliquot (300 μl) using an iodide-sensitive electrode (Orion Research Inc., Boston, Mass.). Relative iodide efflux rate was calculated using the difference between maximum (peak) iodide concentration during stimulation and minimum iodide concentration before stimulation (in μM/min). Data are presented as means±SEM.

Voltage Clamp of CFBE41o− Cell Monolayers

Short-circuit current (Isc) was measured across monolayers in modified Ussing chambers. 1×10⁶ CFBE41o⁻ cells were seeded onto 12-mm fibronectin-coated Snapwell inserts (Corning Incorporated, Life Sciences, New-York, N.Y.) and the apical medium was removed after 24 h. Transepithelial resistance was monitored using an EVOM epithelial voltohmmeter (World Precision Instruments, Sarasota, Fla.) and cells were used when the transepithelial resistance was 300-400 ohms·cm². In some experiments, F508del-CFBE41o⁻ monolayers were incubated at 29° C. or treated with glafenine at 37° C. for 24 h before being mounted in EasyMount chambers and voltage clamped using a VCCMC6 multichannel current-voltage clamp (Physiologic Instruments, San Diego, Calif.). The apical membrane conductance was functionally isolated by permeabilizing the basolateral membrane with 200 μg/ml nystatin and imposing an apical-to-basolateral Cl⁻ gradient. For these experiments the basolateral bathing solution contained (in mM) 1.2 NaCl, 115 Na-gluconate, 25 NaHCO₃, 1.2 MgCl₂, 4 CaCl₂, 2.4, KH₂PO₄, 1.24 K₂HPO₄, 10 glucose (pH 7.4 with NaOH). The CaCl₂ concentration was increased to 4 mM to compensate for the chelation of calcium by gluconate. The apical bathing solution contained (in mM) 115 NaCl, 25 NaHCO₃, 1.2 MgCl₂, 1.2 CaCl₂, 2.4 KH₂PO₄, 1.24 K₂HPO₄, 10 mannitol (pH 7.4 with NaOH). The apical solution contained mannitol instead of glucose to eliminate currents mediated by Na⁺-glucose co-transporter. Successful permeabilization of the basolateral membrane was obvious from the reversal of I_{sc} under these conditions. Solutions were continuously gassed and stirred with 95% O₂-5% CO₂ and maintained at 37° C. Ag/AgCl reference electrodes were used to measure transepithelial voltage and pass current. Pulses (1-mV amplitude, 1 s duration) were imposed at intervals of 90 s to monitor resistance. The voltage clamps were connected to a PowerLab/8SP interface (ADInstruments, Colorado Springs, Colo.) for data collection. CFTR was activated by the addition of 10 μM forskolin+50 μM genistein to the apical bathing solution.

### Ex Vivo and In Vivo Experiments

Glafenine was tested ex vivo and in vivo using ileum from homozygous Δ508-CFTR mice (CFTR^{tm1 Eur}; van Doorninck et al., 1995) and wild-type littermates controls. Mice were 14-17 weeks old, weighed 24-30 g, and were genotyped by standard PCR methods using tail DNA. The mice were kept in a pathogen-free environment in the animal facility at McGill University and fed a high protein diet (SRM-A, Hope Farms, Woerden, NL) modified to contain pork instead of beef. All procedures followed Canadian Institutes of Health Research (CIHR) guidlines and were approved by the faculty Animal Care Committee. For ex vivo experiments, ileal mucosa was stripped of muscle and incubated in William's E-Glutamax medium supplemented with insulin (10 μg/ml), 100 U/ml penicillin, and 100 μg/ml streptomycin and dexamethasone (1.6 ng/ml) containing 0.1% DMSO (control) or DMSO containing glafenine for 5 h. The tissue was then rinsed repeatedly and mounted in Ussing chambers to measure L. For in vivo experiments, mice were feed once per day by gavage with saline containing glafenine (50 mg/kg) or vehicle alone (0.1% DMSO). After 2 days of glafenine treatment the mice were euthanized under CO₂, the intestine was dissected and the short circuit current was measured as described above.

### Salivary Secretion

The procedure followed those recently described by Best & Quinton (Best and Quinton, 2005). Homozygous Δ508-CFTR mice (CFTR^{tm1 Eur}) and wild-type mice of the same strain were 10-12 weeks old and weighed 20-25 g. They were fed once a day by gavage with saline containing glafenine (50 mg/kg) or vehicle alone (0.1% DMSO) for 2 days. The mice were anaesthetized with ketamine and diazepam on the day of the experiment, then pretreated with a subcutaneous injection of 1 mM atropine into the left cheek. Small strips of Whatman filter paper were placed inside the previously injected cheek for ˜4 min to absorb any salivary secretions. A solution containing 100 μM isoprenaline and 1 mM atropine was then injected into the left cheek at the same site to induce secretion at time zero and the filter paper was replaced every minute for 30 minutes. Each piece of filter paper was immediately placed and sealed in a pre-weighed vial and the time of removal was recorded. The rate of salivary secretion per min and total amount were normalized to the mass of the mouse in grams.

### Statistics

All results are expressed as the mean±SEM obtained using N mice. Data sets were compared by analysis of variance (ANOVA) or Student's t-tests using GraphPad Prism version 4. Differences were considered statistically significant when p<0.05. ns: non significant difference, *p<0.05, **p<0.01, ***p<0.001.

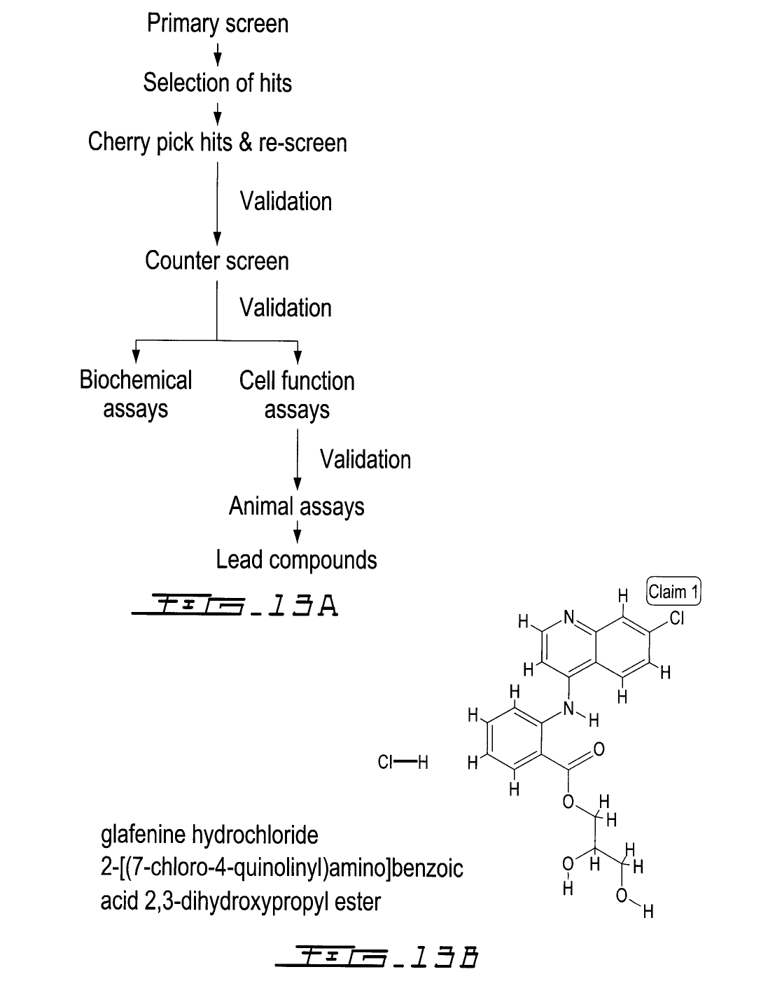

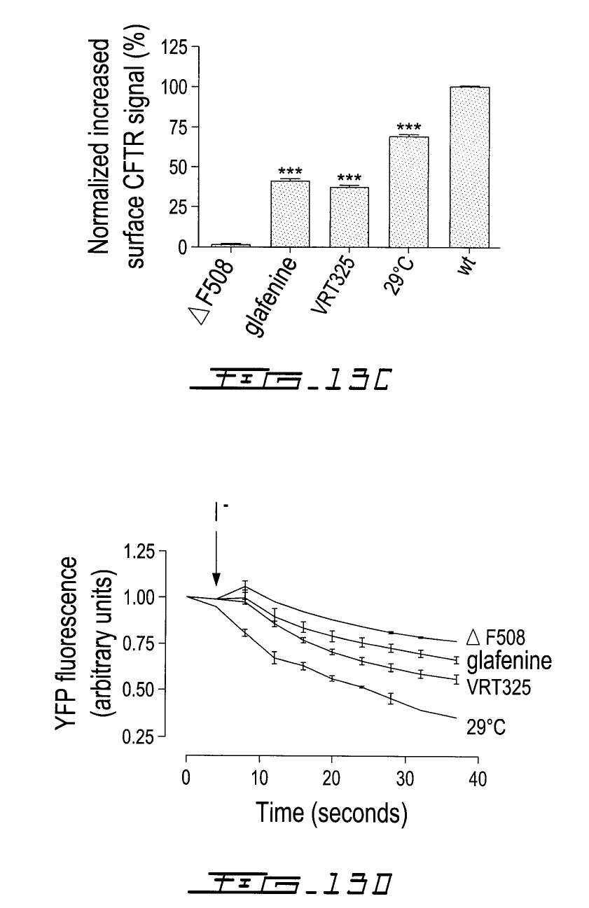

The steps needed to identify and validate hit compounds in a HTS campaign are outlined in FIG. 13A. For the first step, a trafficking assay based on the immunodetection of HA epitopes in the fourth extracellular loop of ΔF508-CFTR was used (Carlile et al., 2007). The primary screen of 1120 compounds in the Prestwick Chemical library yielded 61 positives, identified as having fluorescence that was greater than 1 s.d. above the mean for the plate. These positive compounds were cherry picked and re-tested in duplicate. At this re-screening stage, the intrinsic fluorescence of each positive was measured and those with intrinsic fluorescence were not considered further. Thus, from the original 61 positives, 50 compounds were validated as hits and tested in a counter screen for functional correction using a YFP fluorescence quenching assay. Of those 50 hits, 15 were also positive in the functional assay, quenching 77-91% of the YFP signal. One of the most potent hits validated by this functional assay was glafenine hydrochloride (2-[(7-chloro-4-quinolinyl)amino]benzoic acid 2,3-dihydroxypropyl ester; FIG. 13B), a non-steroidal anti-inflammatory drug that has been used for the relief of pain. According to the trafficking assay glafenine increased ΔF508-CFTR surface expression by 40% increase when compared with ΔF508-CFTR cells treated with vehicle alone and normalized to BHK cells expressing wild-type CFTR, although this is arbitrary since (FIG. 13C). The effects of glafenine were compared with those of the well-established corrector VRT325 under identical conditions (VanGoor et al., 2006). VRT325 caused a similar increase in ΔF508-CFTR cell surface expression (36%), although the level of surface expression was still lower than after temperature correction, or when compared to a representative cell line expressing 3HA-tagged WT-CFTR used for normalization (FIG. 13C).

Effects on trafficking were confirmed using a functional assay (FIG. 13D). Treating cells with 10 μM glafenine for 24 h enhanced the cAMP-stimulated iodide influx in cells expressing ΔF508-CFTR, indicating that functional CFTR at the plasma membrane was increased (FIG. 13D). By comparison, the VRT325 caused a somewhat larger YFP-quenching response (FIG. 13D).

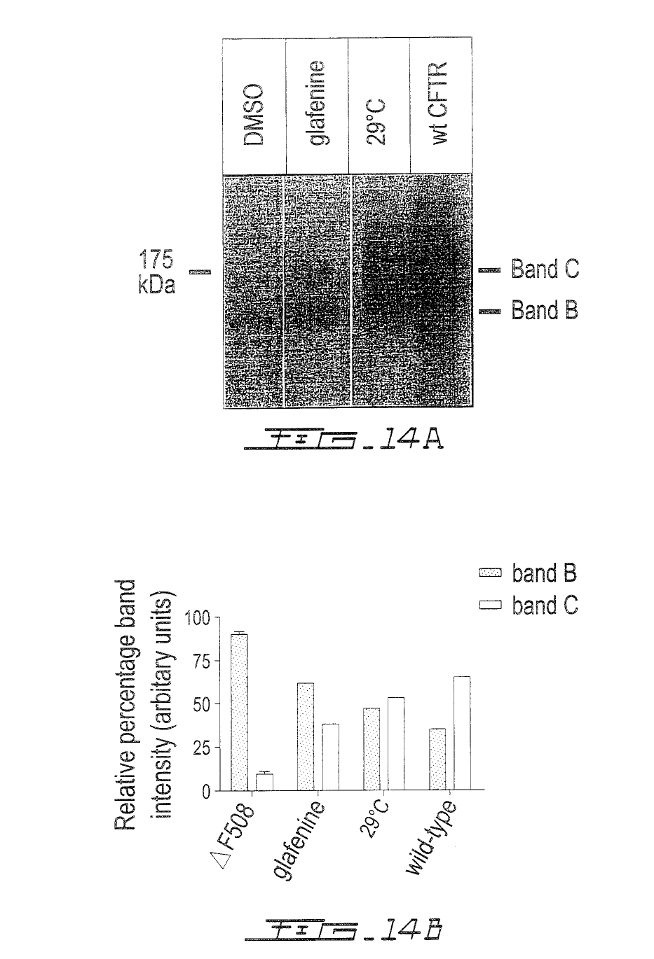

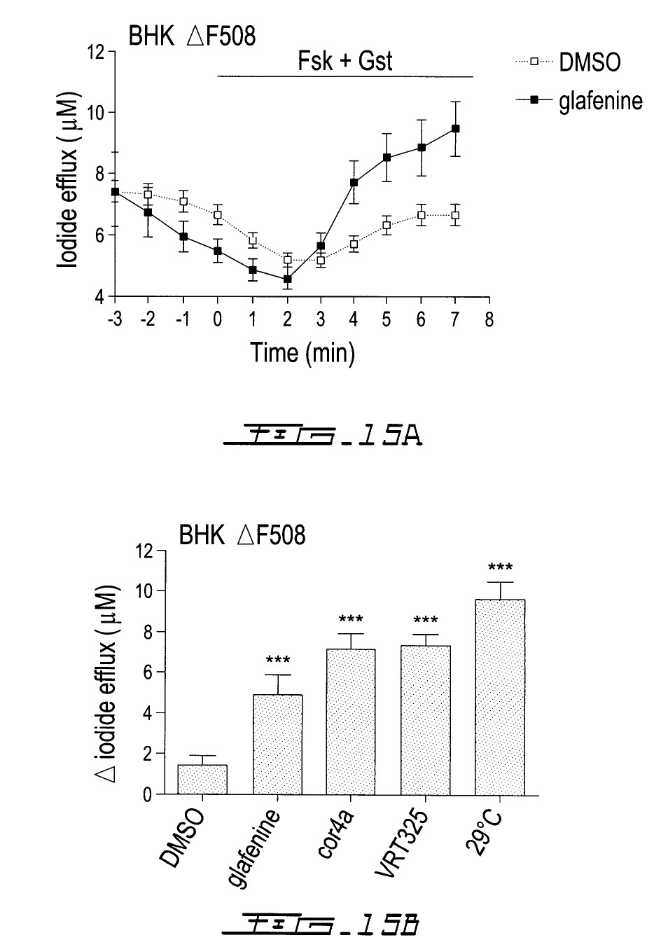

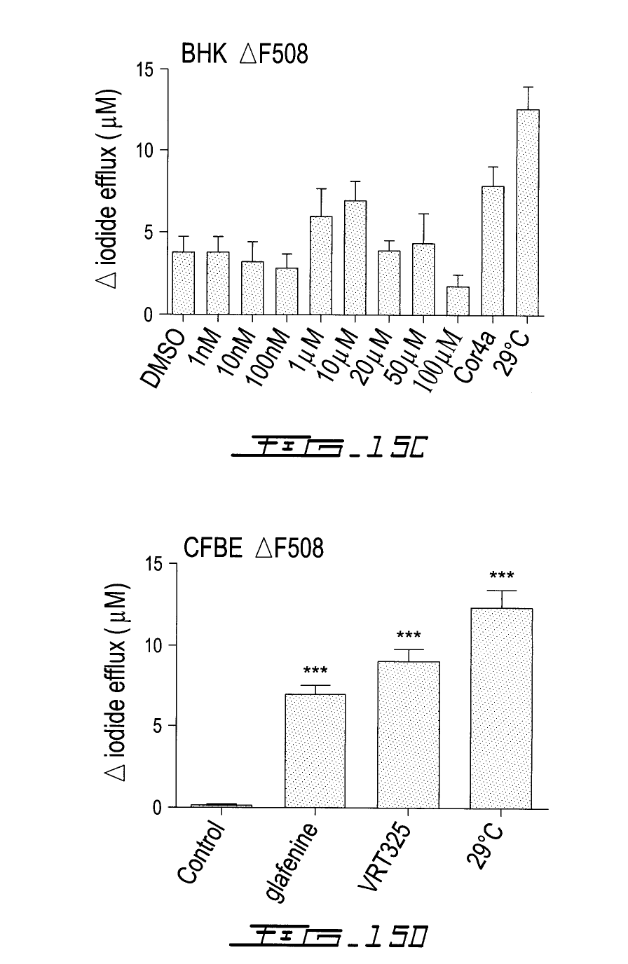

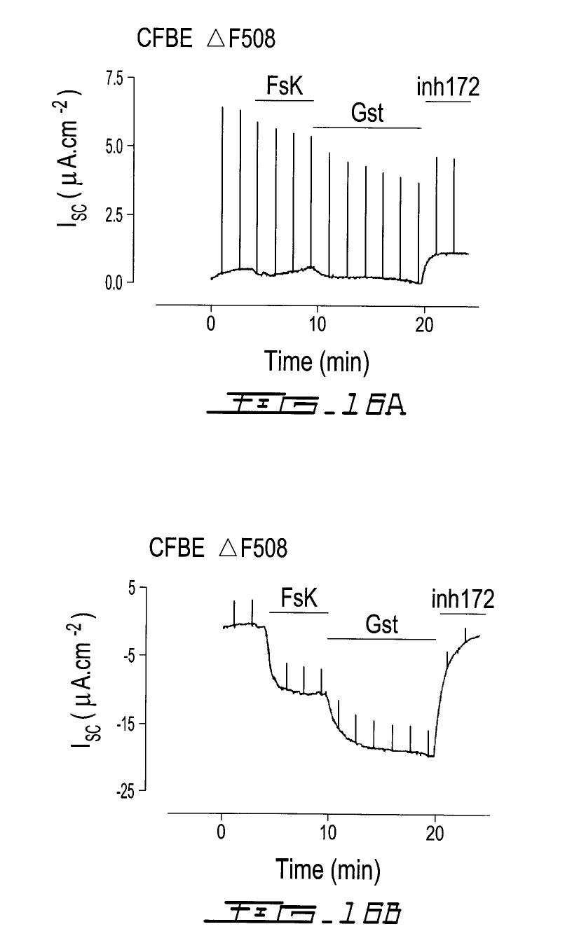

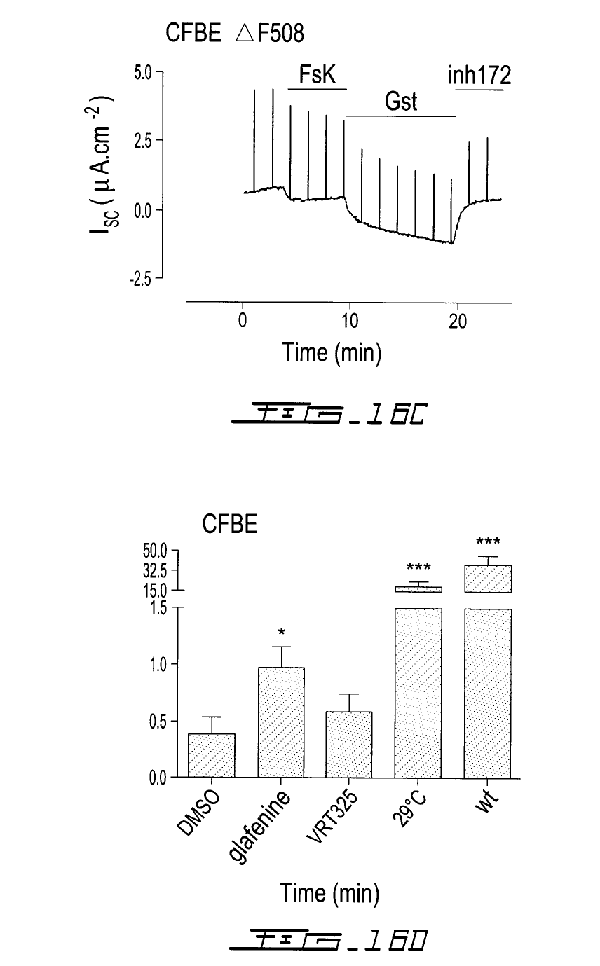

To further validate glafenine as a corrector of CFTR, its effect on protein expression and maturation was analyzed by immunoblotting (FIG. 14A). Maturation of ΔF508-CFTR was confirmed by the appearance of band C in BHK cells treated with 10 μM glafenine for 24 h, consistent with the results of the screening assays (FIG. 14A). As shown in FIG. 14B, approximately 38% of the CFTR signal generated by cells atter treatment with glafenine was the band C glycoform, which represents a 28.4% increase compared to untreated control (ΔF508; FIG. 14B). In BHK cells incubated at 29° C. for 24 h, 53% of the CFTR was the band C glycoform. The next step in the flow chart in FIG. 14A examined the functionality of rescued ΔF508-CFTR using an automated iodide efflux assay, for comparison with the effect of known correctors such as VRT325 and cor4a (FIG. 15). 10 μM glafenine treatment for 24 h restored iodide efflux responses to 10 μM forskolin+50 μM genistein, compared to control cells treated with vehicle alone (FIG. 15A). Consistent with the counter screen using the YFP-quenching fluorescence, glafenine treatment increased the cAMP-stimulated response 3.3-fold, as compared to 4.9-fold or 7-fold change obtained with the correctors, or in cells incubated at low temperature, respectively (FIG. 15B). The concentration dependence of glafenine effects on iodide effluxes was also examined and it was found that 1-10 μM glafenine was required for significant restoration of ΔF508-CFTR function (FIG. 15C). Rescue by glafenine was more striking when tested using the CF (ΔF508/ΔF508) airway epithelial cell line CFBE41o− (FIG. 15D). Finally, glafenine was tested using polarized monolayers of CFBE41o⁻ cells cultured on permeable supports and mounted in Ussing chambers to monitor CFTR-dependent I_{sc}. The basolateral membrane was permeabilized using nystatin to ensure that stimulated I_{sc} was mediated by apical Cl⁻ conductance. FIGS. 16A-C show representative recordings of the I_{sc} obtained from ΔF508-CFBE41o⁻ monolayers that had been incubated with vehicle alone at 37° C. or 29° C., or with 10 μM glafenine at 37° C. for 24 h, respectively. Forskolin and genistein had no effect on the untreated cells maintained at 37° C. (FIG. 16A) but did stimulate current across monolayers that had been incubated at low-temperature (FIG. 16B), and these responses were sensitive to the CFTR channel blocker CFTRᵢₙₕ-172 (10 μM; Ma et al., 2002). Glafenine treatment (10 μM for 24 h) increased the forskolin+genistein-stimulated I_{sc} by about 2.5-fold compared with DMSO controls (FIGS. 16 C, D). The corrected I_{sc} was blocked by CFTRᵢₙₕ-172, evidence that the entire stimulation was mediated by rescued ΔF508-CFTR (FIG. 16C). Correction by glafenine (glafenine; n=9) was greater than that produced by VRT325, which was not different from vehicle controls, however the glafenine-induced I_{sc} was much smaller compared to that induced by low-temperature (29° C.; n=5) or CFBE41o⁻ cells expressing wild-type CFTR (wt; n=14) (FIG. 16D).

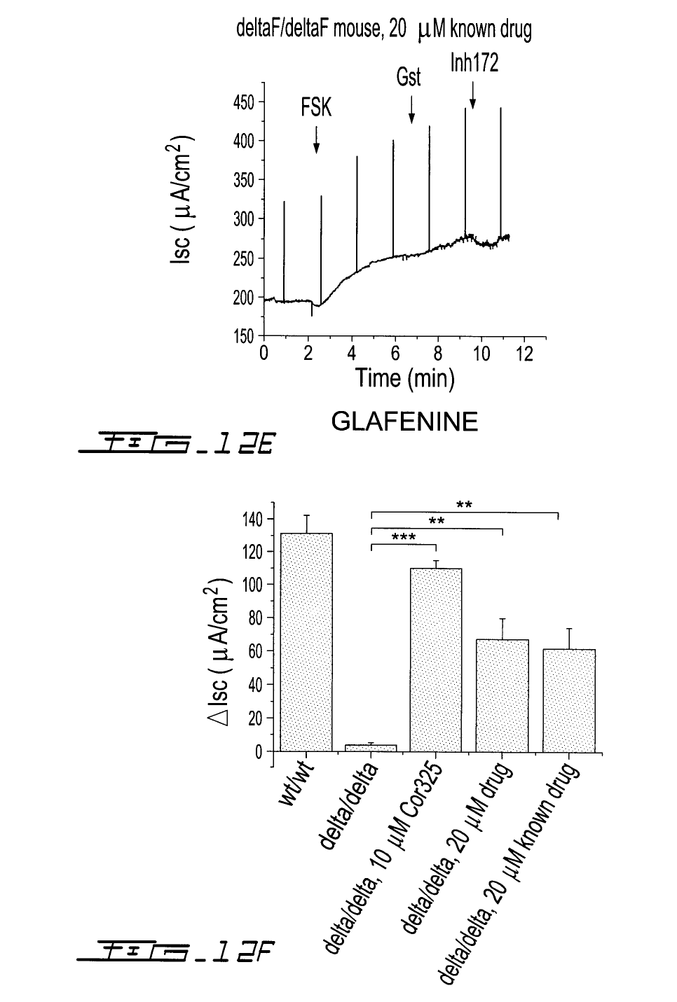

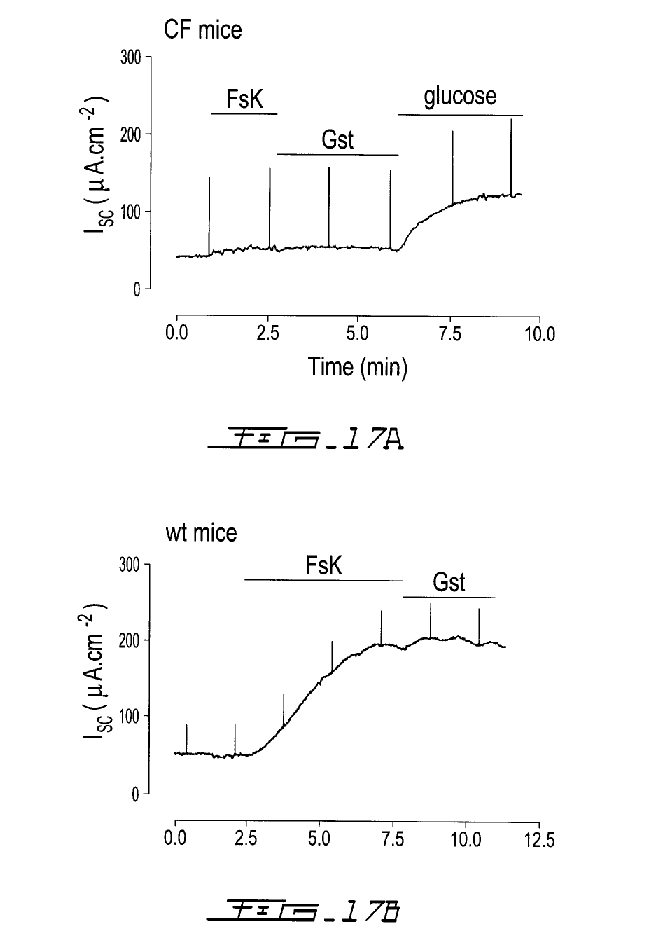

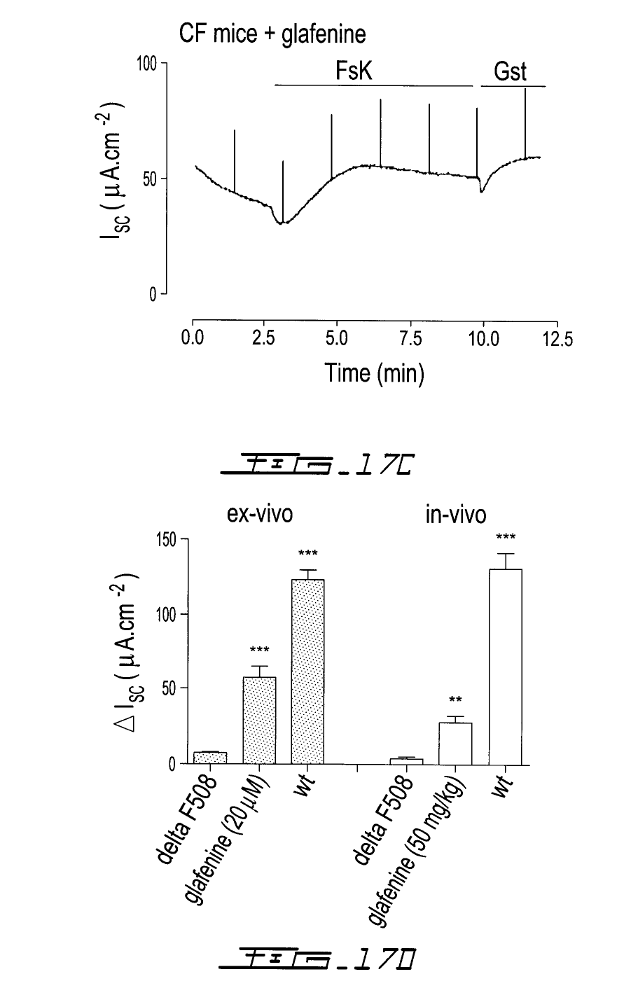

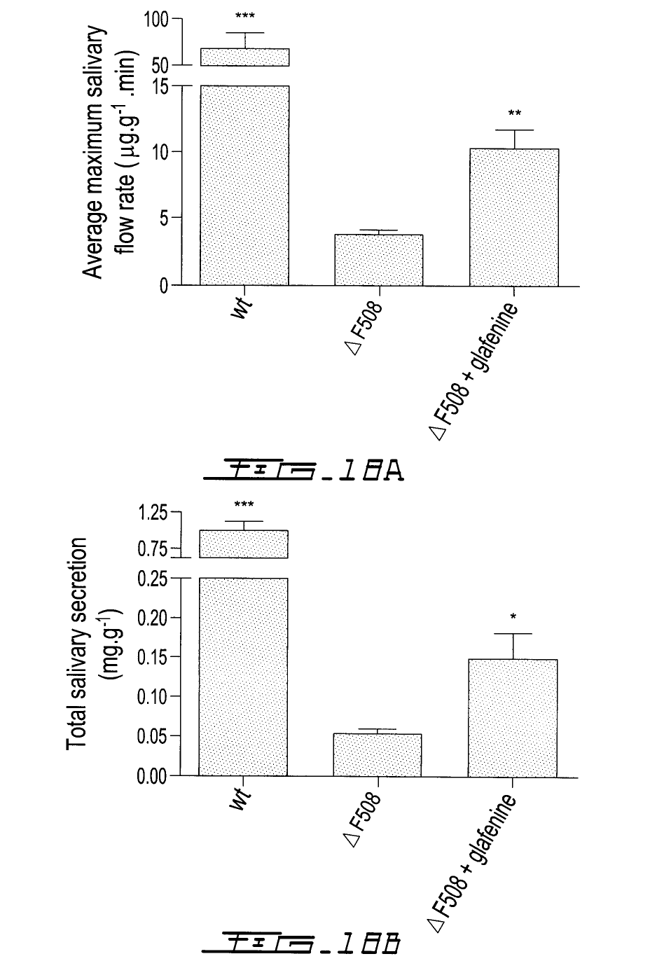

Glafenine effects were studied further using CF mice (see FIG. 13A). Intestinal tissues were isolated from CF mice (i.e. homozygous for ΔF508-CFTR), incubated ex vivo in saline containing 20 μM glafenine for 5-6 h, then examined for their I_{sc} response to forskolin and genistein for comparison with intestines from CF (ΔF508) and non-CF mice (wt). Forskolin+genistein-stimulated currents were ˜12-fold larger when the ileum was treated with glafenine than when treated with vehicle alone (FIGS. 12 and 17A, D and 17B, D, n=5). This increase represents restoration of ˜46% of the secretory response observed in wild-type mice (FIGS. 12 and 17C, D, n=5). To investigate the efficacy of the effect of glafenine in vivo, mice were fed by gavage for 2 days (once per day) with a physiological solution containing 50 mg/kg glafenine. The mice were killed, and the intestine mounted in Ussing chambers to monitor CFTR-dependent I_{sc}. This in vivo treatment increased the forskolin+genistein-stimulated current by ˜7-fold compared to mice fed with saline alone (FIG. 12F and FIG. 17D, grey bar, n=4), and represents a restoration of −21% of the stimulated current obtained using ileum from wild-type mice (FIG. 12F and FIG. 17D, grey bar, n=4). Finally, the efficacy of glafenine in vivo was investigated using the salivary secretion assay developed by Best & Quinton (Best and Quinton, 2005). Mice were fed by gavage with saline alone or saline containing 50 mg/kg glafenine for 2 days, once a day. When stimulated with isoprenaline (in the presence of the atropine), the maximum rate of saliva production by control CF mice was 5% that of WT mice (69.26 μl (g body weight)⁻¹ min⁻¹), and this was increased to 15% in mice treated with glafenine (FIG. 18A). Thus glafenine caused a −2.7-fold increase in the secretory response to β-adrenergic stimulation, which is mediated by CFTR (FIG. 18B).

Taken together, the results from ex vivo and in vivo experiments indicate that glafenine partially corrects defective processing of ΔF508-CFTR in mouse ileum, consistent with the gain of function observed using BHK and the CFBE41 o− human airway epithelial cell line.

Identifying small molecules that correct the processing of CFTR mutants is the first step towards development of an effective therapy for cystic fibrosis (Loo et al., 2005; Pedemonte et al., 2005; Van Goor et al., 2006; Hwang et al., 2007; Carlile et al., 2007; Robert et al., 2008). This was the rationale for screening the Prestwick Library of 1120 bioavailable compounds using the HTS assay of the present invention. The screen identified glafenine as a corrector of CFTR trafficking. Glafenine is an anthranilic acid derivative with analgesic properties which has been used to relieve pain particularly in dentistry since the sixties (Pellegrini et al., 1965). Its ability to correction the misprocessing of CFTR was validated by in vitro studies using concentrations (10 μM) that are achieved clinically in plasma. At 10 μM, glafenine partially corrected ΔF508-CFTR processing and increased its surface expression to ˜40% of that observed for wt-CFTR, comparable to the known corrector VRT325 (van Goor et al., 2006). Treating BHK cells that express ΔF508-CFTR with 10 μM glafenine for 24 h produced a significant increase in the complex glycosylated form of CFTR according to Western-blot analysis. These results are consistent with partial functional correction of ΔF508-CFTR-mediated transport as monitored using two distinct halide flux assays performed in three cell types (BHK, HEK293 and CFBE41o⁻ airway epithelial cells). Ex vivo exposure of CF mouse ileum to glafenine restored ≈46% of the secretory response observed in wild-type mice ileum. Further studies are also needed to establish the mechanism of action of glafenine on ΔF508-CFTR maturation.

When used clinically, the oral dose of glafenine is in the range 600-1200 mg daily or 10 to 30 mg/kg. Feeding CF mice 50 mg glafenine pre kg restored ≈21% of the normal cAMP response in the ileum and =15% of the total salivary response. Taken together, the results of this study suggest glafenine as a potential drug for CF patients. A possible strategy for the use of glafenine may involve the development of hybrid molecules in order to overcome possible side effects of glafenine in CF patients. Hybrid molecules are already in existence for other indications. For example, hybred molecules comprising selective cyclooxygenase inhibitors together with a nitric oxide moiety have been developed to counter the side effects of NSAIDS.

A robust trafficking HTS assay for detecting correctors of ΔF508-CFTR trafficking has thus been developed and validated. Some of the compounds identified have been previously reported to act on CFTR, notably sildenafil, glafenine, chlorzoxazone and diadzein, although trafficking was not known to be a mode of action for the latter two molecules.¹⁴⁻¹⁶ Interestingly, sildenafil was found to act as a trafficking corrector when cells are treated for 24 hours with much lower concentrations than previously reported (10 μM versus 150 μM).¹⁸ Nevertheless, sildenafil treatment at 10 μM did not produce significant iodide efflux despite the partial correction of ΔF508-CFTR trafficking, although function was detected with 1 mM of sildenafil, the concentration used in previous studies.¹⁸ The discrepancy between the concentration needed for rescue and restoration of function is intriguing and is consistent with the diminished responsiveness of ΔF508-CFTR. In addition to sildenafil and glafenine, several other compounds were identified, such as dacthal, glycyrrhizic acid, chloramphenicol and carboplatin, which are novel correctors that have not been previously reported.

CFTR is a cAMP-activated chloride channel, but its activation is also reported to influence many other membrane proteins, and loss of these non-channel effects may lead to sodium hyperabsorption and other abnormalities that contribute to disease symptoms.²⁰ Thus, a primary screen based on restoring trafficking rather than channel activity may reveal correctors that alleviate those other abnormalities yet do not restore channel function. The hits were confirmed in multiple assays as well as by using epithelial cells since drugs may act differently on ΔF508-CFTR trafficking in fibroblasts and epithelial cells.

Although the present invention has been described hereinabove by way of preferred embodiments thereof, and quite specifically with respect to CFTR, it can be modified without departing from the spirit, scope and nature of the subject invention, as defined in the appended claims. In particular, it is contemplated that the assay described herein may be adapted for use in the identification of small molecules that may have therapeutic value in respect of other protein trafficking diseases.

TABLE 1
ΔF508-CFTR correctors identified by highthroughput screening

STRONG HITS

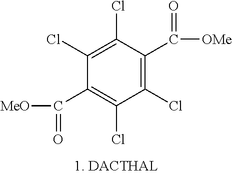

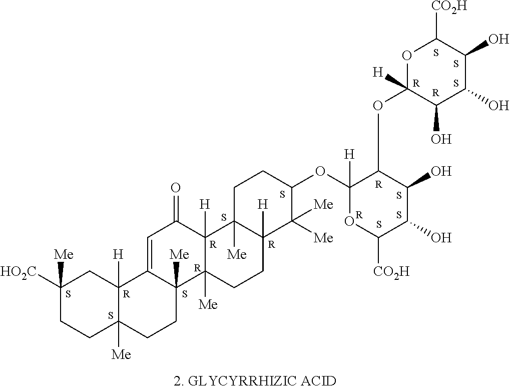

| | |
|---|---|
| 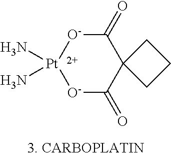 | 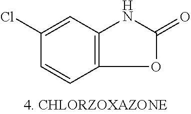 |

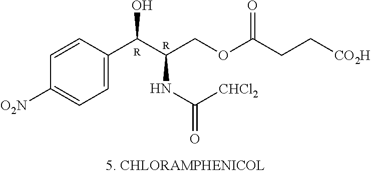

MEDIUM HITS

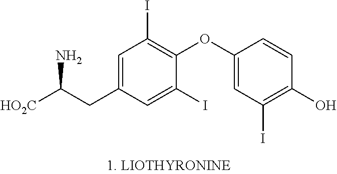

| | |
|---|---|
| 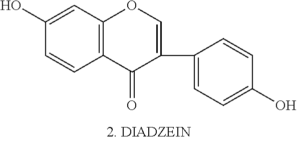 | 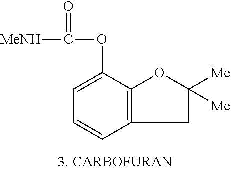 |
| 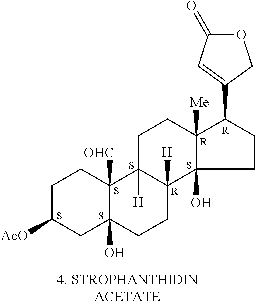 | 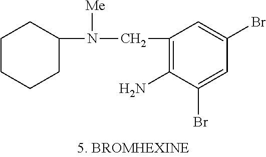 |

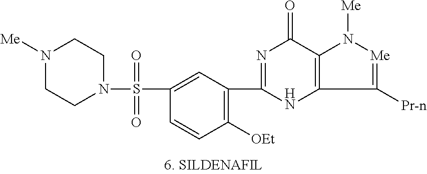

WEAK HITS

| | |
|---|---|
| 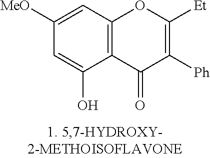 | 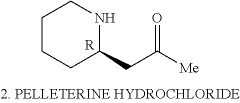 |

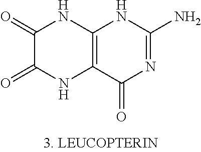

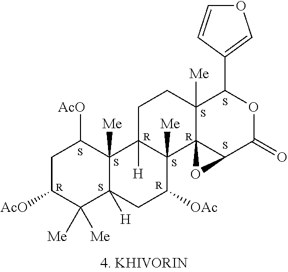

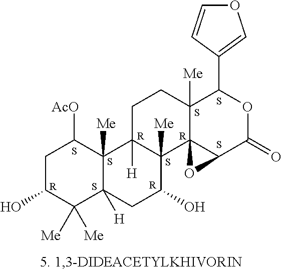

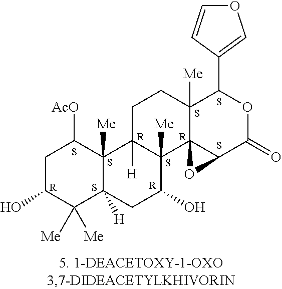

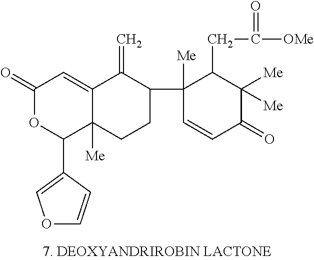

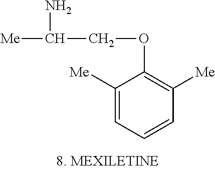

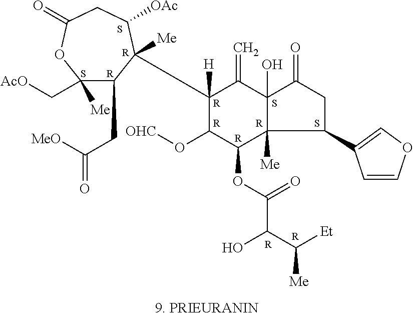

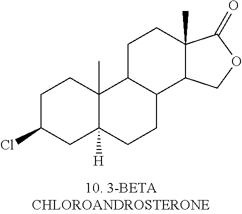

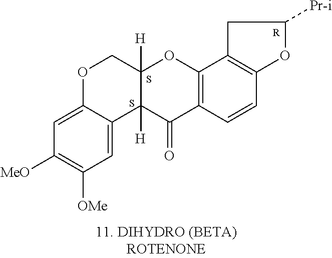

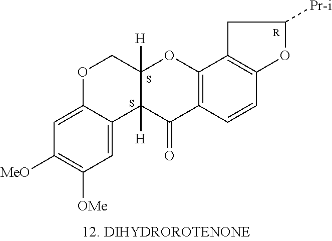

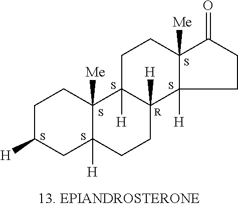

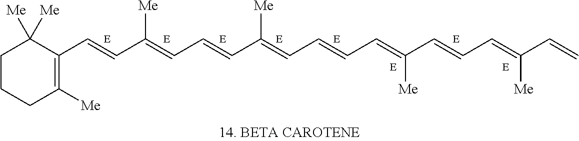

| | |
|---|---|
| 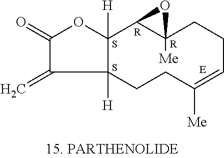 | 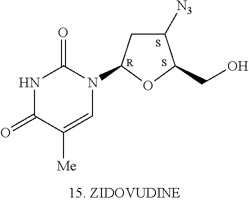 |
| 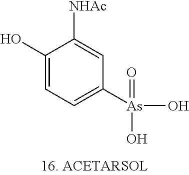 | 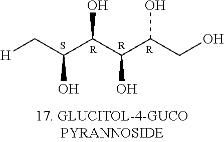 |

TABLE 2
Results for the 50 chosen Prestwick compounds in both the initial corrector assay and the
Functional assay

| | | | | | | % YFP quench |
|---|---|---|---|---|---|---|
| Destination | | Object infos | | | | Compounds |
| CP plate Id | CP well ref | Object Id | Cpd name | hit in screen | Bart's scores | Ordered |
| Cp0019 | A002 | AB-0653229 | Epirizole | W 1.3 | 0/5 | 115 |
| CP0019 | 5002 | AB-0653287 | Hexamethonium dibromide dihydrate | W 1.1 | 1./5 | 98 |
| CP0019 | C002 | AB-0653181 | Dapsone | W 1.1 | 3./5 | 88+ |
| CP0019 | D002 | AR-0653133 | Carbamazenine | W 1.1 | 3./3 | 86 a- |
| CP0019 | E002 | AB-0653035 | Acetohexamide | W 1.0 | 3./3 | 88-+- |
| CP0019 | F002 | AB-0653409 | Thalidomide | M 2.0 | 1./3 | 92 |
| CP0019 | G002 | AB-0653377 | Aminopurine, 6-benzyl | W 1.6 | 2./3 | 89 3- |
| CP0019 | 1-1002 | AB-0653145 | Clemizole hydrochloride | W 1.5 | 3./3 | 84+ |
| CP0019 | A003 | AB-0653471 | Kawain | W 1.1 | 0./5 | 111 |
| CP0019 | 5003 | AB-0653561 | Salbutamol | W 1.0 | 1./5 | 97 |
| CP0019 | C003 | AB-0653548 | Telenzepine dihydrochloride | W 1.3 | 3./5 | 85+ |
| CP0019 | D003 | AB-0653532 | Glafenine hydrochloride | W 1.3 | 3./3 | 80+ |
| CP0019 | E003 | AB-0653525 | Oxybutynin chloride | W 1.2 | 3./3 | 83+ |
| CP0019 | F003 | AB-0653195 | Diethylcarbamazine citrate | W 1.2 | 0./3 | 907 |
| CP0019 | G003 | AB-0653566 | Dropropizine (R, S) | W 1.2 | 0./3 | 92 |
| CP0019 | H003 | AB-0653624 | Dequalinlum dichloride | M 2.2 | 0./3 | 102 |
| CP0019 | A004 | AB-0653507 | Terbutaline hemisulfate | W 1.3 | 0.5/5 | 100 |
| CP0019 | 8004 | AB-0653639 | Amethopterin (R, S) | W 1.2 | 0./5 | 109 |
| CP0019 | C004 | AB-0653616 | Methylergometrine maleate | W 1.1 | 1./5 | 94 |
| CP0019 | D004 | AB-0653465 | Adenosine 5′-monophosphate monohydrate | W 1.1 | 2.5/3 | 78+ |
| CP0019 | E004 | AB-0653642 | Clopamide | W 1.1 | 2.5/3 | 79+ |
| CP0019 | F004 | AB-0653599 | Enoxacin | W 1.0 | 1./3 | 84 ? |
| CP0019 | G004 | AB-0653647 | Clorgyline hydrochloride | W 1.0 | 3./3 | 77+ |
| CP0019 | H004 | AB-0653474 | Lactoblonic acid | W 1.2 | 2./3 | 78+ |
| CP0019 | A005 | AB-0653477 | Cyanocobalamin | W 1.1 | 0./5 | 100 |
| CP0019 | B005 | AB-0653568 | Corticosterone | W 1.1 | 2.5/5 | 90+ |
| CP0019 | C005 | AB-0653293 | Hydrocortisone base | W 1.1 | 0./5 | 94 |
| CP0019 | D005 | AB-0653671 | Carbimazole | W 1.0 | 0./3 | 95 |
| CP0019 | E005 | AB-0653592 | Epiandrosterone | W 1.0 | 1./3 | 93 |
| CP0019 | F005 | AB-0653692 | Tiabendazole | W 1.0 | 0./3 | 907 |
| CP0019 | G005 | AB-0653685 | Nitrofural | W 1.0 | 0./3 | 94 |
| CP0019 | H005 | AB-0653280 | Harmol hydrochloride dihydrate | M 2.5 | 0.5/3 | 92 |
| CP0019 | A006 | AB-0653169 | Cytisine (−) | M 2.0 | 1./5 | 97 |
| CP0019 | 8006 | AB-0653872 | Pramoxine hydrochloride | W 1.2 | 3./5 | 89+ |
| CP0019 | C006 | AB-0653182 | Austricine hydrate | W 1.1 | 1./5 | 91 |
| CP0019 | D006 | AB-0653930 | Estropipate | W 1.0 | 1.5/3 | 83 ? |
| CP0019 | 5006 | AB-0653130 | Merbromin | S 3.6 | 0./3 | 107 |
| CP0019 | F006 | AB-0653814 | Meciofenoxate hydrochloride | W 1.1 | 1./3 | 877 |
| CP0019 | G006 | AB-0653249 | Fenoprofen calcium salt dihydrate | W 1.1 | 1./3 | 88 ? |
| CP0019 | H006 | AB-0653116 | Leflunomide | W 1.0 | 0./3 | 107 |
| CP0019 | A007 | AB-0653874 | Roxithromycin | W 1.2 | 0./5 | 100 |
| CP0019 | 8007 | AB-0653139 | Nafcillin sodium salt monohydrate | W 1.2 | 0.5/5 | 97 |
| CP0019 | C007 | AB-0653154 | Procycildine hydrochloride | W 1.1 | 1./5 | 92 |
| CP0019 | D007 | AB-0654079 | Metyrapone | W 1.4 | 1./3 | 98 |
| CP0019 | E007 | AB-0654006 | S-(+)-Ibuorofen | W 1.1 | 3./3 | 91 9− |
| CP0019 | F007 | AB-0654048 | Flubiprofen | W 1.0 | 1./3 | 98 |
| C.POO 1 9 | G007 | AB-0653062 | Idazoxan hydrochloride | W 1.0 | 0.5/3 | 103 |
| CP0019 | H007 | AB-0653965 | Condelphine | W 1.6 | 1./3 | 97 |
| CP0019 | A008 | AB-065397B | (S)-(−)-Cycloserine | W 1.4 | 0./5 | 98 |
| CP0019 | B008 | AB-0653966 | Oubinidine | W 1.2 | 0./5 | 104 |

nc = 100% pc = 69.2

### LIST OF REFERENCES

1. R. M. Culpepper, S. C. Hebert, T. E. Andreoli, Nephrogenic Diabetes Insipidus 1983, 1867-1867; b) M. C. Sanguinetti, C. Jiang, M. E. Curran, M. T. Keating, Cell, 1995, 81, 299-307.
2. J. R. Riordan, J. M. Rommens, B. Kerem, N. Alon, R. Rozmahel, Z. Grzelczak, J. Zielenski, S. Lok, N. Playsic, J. L. Chou, et al., Science, 1989, 245, 1066-73.
3. N. Pedemonte, G. L. Lukacs, K. Du, E. Caci, O. Zegarra-Moran, L. J. Galietta, A. S. Verkman, J. Clin. Invest., 2005, 115, 2564-71; b) L. V. Galietta, S. Jayaraman, A. S. Verkman, Am. J. Physiol. Cell. Physiol., 2001, 281, C1734-42.
4. Foundation, C.C.F. Report of the Canadian Cystic Fibrosis Patient Data Registry, 2002.
5. C. E. Bear, C. H. Li, N. Kutner, R. J. Bridges, T. J. Jensen, M. Ramjeesingh, J. R. Riordan, Cell, 1992, 68, 809-18.
6. Consortium, T.C.F.G.A. The CFTR mutation database, 2002.
7. M. Denning, L. S. Ostedgaard, M. J. Welsh, J. Cell. Biol., 1992, 118, 551-9; b) C. R. Brown, L. Q. Hong-Brown, J. Biwersi, A. S. Verkman, W. J. Welch, Cell Stress Chaperones, 1996, 1, 117-25.
8. A. Swiatecka-Urban, A. Brown, S. Moreau-Marquis, J. Renuka, B. Coutermarsh, R. Barnaby, K. H. Karlson, T. R. Flotte, M. Fukuda, G. M. Langford, B. A. Stanton, J. Biol. Chem., 2005, 280, 36762-72.
9. S. L. Farmen, P. H. Karp, P. Ng, D. J. Palmer, D. R. Koehler, J. Hu, A. L. Beaudet, J. Zabner, M. J. Welsh, Am. J. Physiol. Lung. Cell. Mol. Physiol., 2005, 289, L1123-30.
10. M. Howard, M. D. DuVall, D. C. Devor, J. Y. Dong, K. Henze, R. A. Frizzell, Am. J. Physiol., 1995, 269, C1565-76.
11. K. Kunzelmann, E. M. Schwiebert, P. L. Zeitlin, W. L. Kuo, B. A. Stanton, D. C. Gruenert, Am. J. Respir. Cell. Mot Biol., 1993, 8, 522-9.
12. X. Wu, J. K. Wakefield, H. Liu, H. Xiao, R. Kralovics, J. T. Prchal, J. C. Kappes, Mol. Ther., 2000, 2, 47-55.
13. Z. Bebok, J. F. Collawn, J. Wakefield, W. Parker, Y. Li, K. Varga, E. J. Sorscher, J. P. Clancy, J. Physiol., 2005, 569, 601-15.
14. L. Gao, J. R. Broughman, T. Iwamoto, J. M. Tomich, C. J. Venglarik, H. J. Forman, Am. J. Physiol. Lung Cell. Mol. Physiol., 2001, 281, L24-30.
15. B. R. Cobb, L. Fan, T. E. Kovacs, E. J. Sorscher, J. P. Clancy, Am. J. Respir. Cell. Mol. Biol., 2003, 29, 410-8.
16. K. Obayashi, M. Hone, T. Washizuka, T. Nishimoto, S. Sasayama, Pflugers Arch., 1999, 438, 269-77.
17. a) P. S. Pedersen, E. W. Flensborg, S. G. Johnsen, Acta Paediatr. Scand., 1981, 70, 507-12; b) P. H. Klaren, P. H. Looijmans, J. Cyst. Fibros., 2004, 3, 125-8; c) I. Hasegawa, N. Niisato, Y. Iwasaki, Y. Marunaka, Biochem. Biophys. Res. Commun., 2006, 343, 475-82; d) A. V. deCarvalho, C. P. Ndi, A. Tsopmo, P. Tane, J. Ayafor, J. D. Connolly, J. L. Teem, Mol. Med., 2002, 8, 75-87.
18. R. L. Dormer, C. M. Harris, Z. Clark, M. M. Pereira, I. J. Doull, C. Norez, F. Becq, M. A. McPherson, Thorax, 2005, 60, 55-9.
19. J. H. Zhang, T. D. Chung, K. R. Oldenburg, J. Biomol. Screen., 1999, 4, 67-73.
20. a) C. Li, A. P. Naren, Pharmacol. Ther., 2005, 108, 208-23; b) P. Huang, E. Gilmore, P. Kultgen, P. Barnes, S. Milgram, M. J. Stuffs, Proc. Am. Thorac. Soc., 2004, 1, 33-7.

1-19. (canceled)

20. A method for identifying small molecules that correct the biosynthetic arrest of mutated cystic fibrosis transmembrane conductance regulator (CFTR), wherein said method comprises:
(A) providing a cell expressing the mutated CFTR;
(B) contacting the cell with a test compound; and
(C) determining whether the compound affects the expression of the mutated CFTR within the cell by measuring the appearance of the mutated CFTR at the cell surface.

21. The method of claim 20, wherein said mutated CFTR is ΔF508-CFTR.

22. The method of claim 21, wherein said mutated CFTR is tagged.

23. The method of claim 22, wherein said tag is comprised of three HA tags inserted into the fourth extracellular loop of CFTR.

24. The method of claim 20, wherein said method is used as a high throughput screen.

25. A method for treating or alleviating the symptoms of cystic fibrosis in a patient, wherein said method comprises administering to said patient a compound selected from the group consisting of: dacthal, glycyrrhizic acid, carboplatin, chlorzoxazone, chloramphenicol, liotyronine, diadzein, carbofuran, storphanthidin acetate, bromhexine, sildenafil, 5,7-hydroxy-2-methoisoflavone, pelleterine hydrochloride, leucopterin, khivorin, 1,3-dideacetylkhivorin, 1-deacetoxy-1-oxo-3,7,-dideacetylkhivorin, deoxy-andrirobin lactone, mexiletine, prieuranin, 3-beta chloroandrosterone, dihydro(beta)rotenone, dihydrorotenone, epiandrosterone, beta carotene, parthenolide, zidovudine, acetarsol, glucitol-4-gucopyrannoside, dapsone, clemizole hydrochloride, lactobionic acid, corticosterone, pramoxine hydrochloride, 6-benzyl amino purine, iacetohexamide, ibuprofen, glafenine hydrochloride, telenzepine hydrochloride, AMP monohydrate chloride, clopamide, n-methyl-n-parachloro propyl amine hydrochloride, oxybutinin chloride and carbamazepine.

26. The method of claim 25, wherein said compound is selected from the group consisting of: sildenafil, dapsone, clemizole hydrochloride, lactobionic acid, corticosterone, pramoxine hydrochloride, 6-benzyl amino purine, iacetohexamide, ibuprofen, glafenine hydrochloride, telenzepine hydrochloride, AMP monohydrate chloride, clopamide, n-methyl-n-parachloro propyl amine hydrochloride, oxybutinin chloride and carbamazepine.

27. The method of claim 26, wherein said compound is sildenafil or glafenine hydrochloride.

28. A method for treating or alleviating the symptoms of chronic obstructive pulmonary disease (CODP) in a patient, wherein said method comprises administering to said patient a compound selected from the group consisting of: dacthal, glycyrrhizic acid, carboplatin, chlorzoxazone, chloramphenicol, liotyronine, diadzein, carbofuran, storphanthidin acetate, bromhexine, sildenafil, 5,7-hydroxy-2-methoisoflavone, pelleterine hydrochloride, leucopterin, khivorin, 1,3-dideacetylkhivorin, 1-deacetoxy-1-oxo-3,7,-dideacetylkhivorin, deoxyandrirobin lactone, mexiletine, prieuranin, 3-beta chloroandrosterone, dihydro(beta)rotenone, dihydrorotenone, epiandrosterone, beta carotene, parthenolide, zidovudine, acetarsol, glucitol-4-gucopyrannoside, dapsone, clemizole hydrochloride, lactobionic acid, corticosterone, pramoxine hydrochloride, 6-benzyl amino purine, iacetohexamide, ibuprofen, glafenine hydrochloride, telenzepine hydrochloride, AMP monohydrate chloride, clopamide, n-methyl-n-parachloro propyl amine hydrochloride, oxybutinin chloride and carbamazepine.

29. The method of claim 28, wherein said compound is selected from the group consisting of: sildenafil, dapsone, clemizole hydrochloride, lactobionic acid, corticosterone, pramoxine hydrochloride, 6-benzyl amino purine, iacetohexamide, ibuprofen, glafenine hydrochloride, telenzepine hydrochloride, AMP monohydrate chloride, clopamide, n-methyl-n-parachloro propyl amine hydrochloride, oxybutinin chloride and carbamazepine.

30. The method of claim 29, wherein said compound is sildenafil or glafenine hydrochloride.

31. The method of claim 28, wherein said COPD is acute or chronic bronchitis, emphysema, pneumoconiosis or is caused by pulmonary neoplasms.

32. A method for treating or alleviating the symptoms of nephrogenic diabetis insipidus (NDI) in a patient, wherein said method comprises administering to said patient a compound selected from the group consisting of: dacthal, glycyrrhizic acid, carboplatin, chlorzoxazone, chloramphenicol, liotyronine, diadzein, carbofuran, storphanthidin acetate, bromhexine, sildenafil, 5,7-hydroxy-2-methoisoflavone, pelleterine hydrochloride, leucopterin, khivorin, 1,3-dideacetylkhivorin, 1-deacetoxy-1-oxo-3,7,-dideacetylkhivorin, deoxyandrirobin lactone, mexiletine, prieuranin, 3-beta chloroandrosterone, dihydro(beta)rotenone, dihydrorotenone, epiandrosterone, beta carotene, parthenolide, zidovudine, acetarsol, glucitol-4-gucopyrannoside, dapsone, clemizole hydrochloride, lactobionic acid, corticosterone, pramoxine hydrochloride, 6-benzyl amino purine, iacetohexamide, ibuprofen, glafenine hydrochloride, telenzepine hydrochloride, AMP monohydrate chloride, clopamide, n-methyl-n-parachloro propyl amine hydrochloride, oxybutinin chloride and carbamazepine.

33. The method of claim 32, wherein said compound is selected from the group consisting of: sildenafil, dapsone, clemizole hydrochloride, lactobionic acid, corticosterone, pramoxine hydrochloride, 6-benzyl amino purine, iacetohexamide, ibuprofen, glafenine hydrochloride, telenzepine hydrochloride, AMP monohydrate chloride, clopamide, n-methyl-n-parachloro propyl amine hydrochloride, oxybutinin chloride and carbamazepine.

34. The method of claim 33, wherein said compound is sildenafil or glafenine hydrochloride.

35. A method for treating or alleviating in a patient the symptoms of cystic fibrosis, chronic obstructive pulmonary disease (COPD) or nephrogenic diabetis insipidus (NDI) wherein said method comprises administering to said patient telenzepine hydrochloride, AMP monohydride chloride, pramoxine hydrochloride or lactobionic acid.

36. The method of claim 35, wherein said COPD is acute or chronic bronchitis, emphysema, pneumoconiosis or is caused by pulmonary neoplasms.

37. A method for monitoring the effect of a compound on the trafficking of a mutated cystic fibrosis transmembrane conductance regulator (CFTR) to the plasma membrane, wherein said mutated CFTR gene is tagged in order to track its displacement within a cell.
